(19) United States
(12) Patent Application Publication
(10) Pub. No.: US 2014/0213576 A1
(43) Pub. Date: Jul. 31, 2014

(54) NOVEL COMPOUND USEFUL FOR THE TREATMENT OF DEGENERATIVE AND INFLAMMATORY DISEASES

(71) Applicant: GALAPAGOS NV, Mechelen (BE)

(72) Inventor: Christel Jeanne Marie MENET, Mechelen (BE)

(73) Assignee: GALAPAGOS NV, Mechelen (BE)

(21) Appl. No.: 14/154,245

(22) Filed: Jan. 14, 2014

Related U.S. Application Data
(60) Provisional application No. 61/753,482, filed on Jan. 17, 2013.

(30) Foreign Application Priority Data
Aug. 6, 2013 (GB) 1314015.7

(51) Int. Cl. C07D 471/04; A61K 45/06; A61K 31/444

(52) U.S. Cl. CPC C07D 471/04; A61K 31/444; A61K 45/06
USPC 514/210.21; 546/118

(57) ABSTRACT

A novel compound according to Formula I, able to inhibit JAK as disclosed, this compound may be prepared as a pharmaceutical composition, and may be used for the prevention and treatment of a variety of conditions in mammals including humans, including by way of non-limiting example, allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

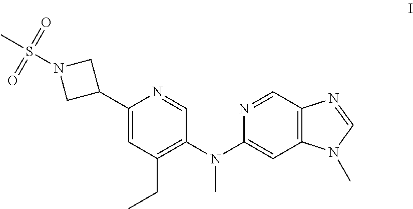 [SMILES: CCc1cc(C2CN(S(C)(=O)=O)C2)ncc1N(C)c1cc2c(cn1)ncn2C]

### RELATED APPLICATIONS

The present application claims the benefit under 35 U.S.C. §119 of U.S. Provisional Application No. 61/753,482, filed Jan. 17, 2013, and G.B. Application No. 1314015.7, filed Aug. 6, 2013, the contents of each of which are hereby incorporated by reference in their entireties.

### FIELD OF THE INVENTION

The present invention relates to a compound that is an inhibitor of JAK, a family of tyrosine kinases that are involved in allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. In particular, the compound of the invention inhibits JAK1 and/or JAK2, and more particularly the compound of the invention inhibits JAK1. The present invention also provides methods for the production of the compound of the invention, pharmaceutical compositions comprising the compounds of the invention, methods for the prevention and/or treatment of diseases involving allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons by administering the compound of the invention.

Janus kinases (JAKs) are cytoplasmic tyrosine kinases that transduce cytokine signaling from membrane receptors to STAT transcription factors. Four JAK family members are described, JAK1, JAK2, JAK3 and TYK2. Upon binding of the cytokine to its receptor, JAK family members auto- and/or transphosphorylate each other, followed by phosphorylation of STATs that then migrate to the nucleus to modulate transcription. JAK-STAT intracellular signal transduction serves the interferons, most interleukins, as well as a variety of cytokines and endocrine factors such as EPO, TPO, GH, OSM, LIF, CNTF, GM-CSF and PRL (Vainchenker W. et al. (2008)).

The combination of genetic models and small molecule JAK inhibitor research revealed the therapeutic potential of several JAKs. JAK3 is validated by mouse and human genetics as an immune-suppression target (O'Shea J. et al. (2004)). JAK3 inhibitors were successfully taken into clinical development, initially for organ transplant rejection but later also in other immuno-inflammatory indications such as rheumathoid arthritis (RA), psoriasis and Crohn's disease (http://clinicaltrials.gov/).

TYK2 is a potential target for immuno-inflammatory diseases, being validated by human genetics and mouse knock-out studies (Levy D. and Loomis C. (2007)).

JAK1 is a target in the immuno-inflammatory disease area. JAK1 heterodimerizes with the other JAKs to transduce cytokine-driven pro-inflammatory signaling. Therefore, inhibition of JAK1 is of interest for immuno-inflammatory diseases with pathology-associated cytokines that use JAK1 signaling, such as IL-6, IL-4, IL-5, IL-12, IL-13, IL-23, or IFNgamma, as well as for other diseases driven by JAK-mediated signal transduction.

### BACKGROUND OF THE INVENTION

The degeneration of cartilage is the hallmark of various diseases, among which rheumatoid arthritis and osteoarthritis are the most prominent. Rheumatoid arthritis (RA) is a chronic joint degenerative disease, characterized by inflammation and destruction of the joint structures. When the disease is unchecked, it leads to substantial disability and pain due to loss of joint functionality and even premature death. The aim of an RA therapy, therefore, is not only to slow down the disease but to attain remission in order to stop the joint destruction. Besides the severity of the disease outcome, the high prevalence of RA (˜0.8% of adults are affected worldwide) means a high socio-economic impact. (For reviews on RA, we refer to Smolen and Steiner (2003); Lee and Weinblatt (2001); Choy and Panayi (2001); O'Dell (2004) and Firestein (2003)).

JAK1 and JAK2 are implicated in intracellular signal transduction for many cytokines and hormones. Pathologies associated with any of these cytokines and hormones can be ameliorated by JAK1 and JAK2 inhibitors. Hence, several allergy, inflammation and autoimmune disorders might benefit from treatment with compounds described in this invention including rheumatoid arthritis, systemic lupus erythematosis, juvenile idiopathic arthritis, osteoarthritis, asthma, chronic obstructive pulmonary disease COPD, tissue fibrosis, eosinophilic inflammation, eosophagitis, inflammatory bowel diseases (e.g. Crohn's, ulcerative colitis), transplantation, graft-versus-host disease, psoriasis, myositis, multiple sclerosis (Kopf et al., 2010).

Osteoarthritis (also referred to as OA, or wear-and-tear arthritis) is the most common form of arthritis and is characterized by loss of articular cartilage, often associated with hypertrophy of the bone and pain. For an extensive review on osteoarthritis, we refer to Wieland et al. (2005).

Osteoarthritis is difficult to treat. At present, no cure is available and treatment focuses on relieving pain and preventing the affected joint from becoming deformed. Common treatments include the use of non-steroidal anti-inflammatory drugs (NSAIDs). Although dietary supplements such as chondroitin and glucosamine sulphate have been advocated as safe and effective options for the treatment of osteoarthritis, a recent clinical trial revealed that both treatments did not reduce pain associated with osteoarthritis. (Clegg et al., 2006).

Stimulation of the anabolic processes, blocking catabolic processes, or a combination of these two, may result in stabilization of the cartilage, and perhaps even reversion of the damage, and therefore prevent further progression of the disease. Therapeutic methods for the correction of the articular cartilage lesions that appear during the osteoarthritic disease have been developed, but so far none of them have been able to mediate the regeneration of articular cartilage in situ and in vivo. Taken together, no disease modifying osteoarthritic drugs are available.

Vandeghinste et al. (WO 2005/124342) discovered JAK1 as a target whose inhibition might have therapeutic relevance for several diseases including OA. Knockout of the JAK1 gene in mice demonstrated that JAK1 plays essential and non-redundant roles during development: JAK1−/− mice died within 24 h after birth and lymphocyte development was severely impaired. Moreover, JAK1−/− cells were not, or less, reactive to cytokines that use class II cytokine receptors, cytokine receptors that use the gamma-c subunit for signaling and the family of cytokine receptors that use the gp130 subunit for signaling (Rodig et al., 1998).

Various groups have implicated JAK-STAT signaling in chondrocyte biology. Li et al. (2001) showed that Oncostatin M induces MMP and TIMP3 gene expression in primary chondrocytes by activation of JAK/STAT and MAPK signaling pathways. Osaki et al. (2003) showed that interferon-gamma mediated inhibition of collagen II in chondrocytes involves JAK-STAT signaling. IL1-beta induces cartilage catabolism by reducing the expression of matrix components, and by inducing the expression of collagenases and inducible nitric oxide synthase (NOS2), which mediates the production of nitric oxide (NO). Otero et al., (2005) showed that leptin and IL1-beta synergistically induced NO production or expression of NOS2 mRNA in chondrocytes, and that that was blocked by a JAK inhibitor. Legendre et al. (2003) showed that IL6/IL6 Receptor induced downregulation of cartilage-specific matrix genes collagen II, aggrecan core and link protein in bovine articular chondrocytes, and that this was mediated by JAK/STAT signaling. Therefore, these observations suggest a role for JAK kinase activity in cartilage homeostasis and therapeutic opportunities for JAK kinase inhibitors.

JAK family members have been implicated in additional conditions including myeloproliferative disorders (O'Sullivan et al, 2007, Mol Immunol 44(10):2497-506), where mutations in JAK2 have been identified. This indicates that inhibitors of JAK in particular JAK2 may also be of use in the treatment of myeloproliferative disorders. Additionally, the JAK family, in particular JAK1, JAK2 and JAK3, has been linked to cancers, in particular leukaemias e.g. acute myeloid leukaemia (O'Sullivan et al, 2007, Mol. Immunol. 44(10):2497-506; Xiang et al., 2008, “Identification of somatic JAK1 mutations in patients with acute myeloid leukemia” Blood First Edition Paper, prepublished online Dec. 26, 2007; DOI 10.1182/blood-2007-05-090308) and acute lymphoblastic leukaemia (Mullighan et al, 2009) or solid tumours e.g. uterine leiomyosarcoma (Constantinescu et al., 2007, Trends in Biochemical Sciences 33(3): 122-131), prostate cancer (Tam et al., 2007, British Journal of Cancer, 97, 378-383). These results indicate that inhibitors of JAK, in particular of JAK1 and/or JAK2, may also have utility in the treatment of cancers (leukaemias and solid tumours e.g. uterine leiomyosarcoma, prostate cancer).

In addition, Castleman's disease, multiple myeloma, mesangial proliferative glomerulonephritis, psoriasis, and Kaposi's sarcoma are likely due to hypersecretion of the cytokine IL-6, whose biological effects are mediated by intracellular JAK-STAT signaling (Tetsuji Naka, Norihiro Nishimoto and Tadamitsu Kishimoto, Arthritis Res 2002, 4 (suppl 3):5233-5242). This result shows that inhibitors of JAK may also find utility in the treatment of said diseases.

The current therapies are not satisfactory and therefore there remains a need to identify further compounds that may be of use in the treatment of allergy, inflammatory and auto-immune disorders, proliferative disorders and degenerative joint diseases, e.g. osteoarthritis, rheumatoid arthritis and osteoporosis, in particular osteoarthritis and rheumatoid arthritis. The present invention therefore provides compounds, methods for their manufacture and pharmaceutical compositions comprising the compound of the invention together with a suitable pharmaceutical carrier. The present invention also provides for the use of the compound of the invention in the preparation of a medicament for the treatment of allergy, inflammatory and auto-immune disorders, proliferative disorders and degenerative joint diseases, e.g. osteoarthritis, and rheumatoid arthritis, in particular rheumatoid arthritis.

### SUMMARY OF THE INVENTION

The present invention is based on the discovery that the compound of the invention is able to act as an inhibitor of JAK and that it is useful for the treatment of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. In a specific aspect the compound of the invention is an inhibitor of JAK1 and/or JAK2. In a more specific aspect the compound of the invention is an inhibitor of JAK1. The present invention also provides methods for the production of this compound, pharmaceutical compositions comprising this compound and methods for treating allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons by administering the compound of the invention.

Accordingly, in a first aspect of the invention, the compound of the invention is provided having a Formula (I):

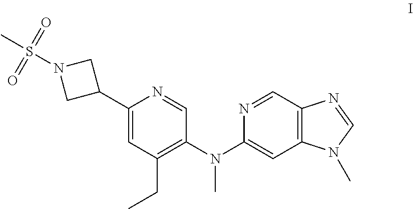 [SMILES: CCc1cc(C2CN(S(C)(=O)=O)C2)ncc1N(C)c1cc2c(cn1)ncn2C]

In a particular embodiment the compound of the invention is an inhibitor of JAK1 and/or JAK2. In a more particular embodiment, the compound of the invention inhibits JAK1 with a selectivity vs the other JAK family members of at least 10 fold. In a most particular embodiment, the compound of the inhibition is a selective JAK1 inhibitor.

In a further aspect, the present invention provides pharmaceutical compositions comprising the compound of the invention, and a pharmaceutical carrier, excipient or diluent. Moreover, the compound of the invention, useful in the pharmaceutical compositions and treatment methods disclosed herein, is pharmaceutically acceptable as prepared and used. In this aspect of the invention, the pharmaceutical composition may additionally comprise further active ingredients suitable for use in combination with the compound of the invention.

In a further aspect, the invention provides the compound of the invention or a pharmaceutical composition comprising the compound of the invention for use as a medicament. In a specific embodiment, said pharmaceutical composition additionally comprises a further active ingredient.

In a further aspect of the invention, this invention provides a method of treating a mammal susceptible to or afflicted with a condition from among those listed herein, and particularly, such condition as may be associated with aberrant JAK activity, e.g. allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, which method comprises administering an effective amount of the pharmaceutical composition or compound of the invention as described herein. In a specific embodiment the condition is associated with aberrant JAK1 and/or JAK2 activity. In a more specific embodiment the condition is associated with aberrant JAK1 activity.

In a further aspect, the present invention provides the compound of the invention for use in the treatment or prophylaxis of a condition selected from those listed herein, particularly such conditions as may be associated with aberrant JAK activity, e.g. allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

In yet another method of treatment aspect, this invention provides a method for treating a mammal susceptible to or afflicted with a condition that is causally related to abnormal JAK activity as described herein, and comprises administering an effective condition-treating or condition-preventing amount of the pharmaceutical composition or the compound of the invention described herein. In a specific aspect the condition is causally related to abnormal JAK1 and/or JAK2 activity. In a more specific aspect the condition is causally related to abnormal JAK1 activity.

In a further aspect, the present invention provides the compound of the invention, or a pharmaceutical composition comprising the compound of the invention, for use as a medicament.

In a further aspect, the present invention provides the compound of the invention for use in the treatment or prophylaxis of a condition that is causally related to abnormal JAK activity.

In additional aspects, this invention provides methods for synthesizing the compound of the invention, with representative synthetic protocols and pathways disclosed later on herein.

Accordingly, it is a principal object of this invention to provide a novel compound, which can modify the activity of JAK and thus prevent or treat any conditions that may be causally related thereto. In a specific aspect the compound of the invention modulate the activity of JAK1 and/or JAK2. In a more specific aspect the compound of the invention modulate the activity of JAK1.

It is a further object of this invention to provide a compound that can treat or alleviate conditions or symptoms of same, such as allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, that may be causally related to the activity of JAK, in particular JAK1 and/or JAK2, and more particularly JAK 1.

A still further object of this invention is to provide a pharmaceutical composition that may be used in the treatment or prophylaxis of a variety of conditions, including the diseases associated with JAK activity such as allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. In a specific embodiment the disease is associated with JAK1 and/or JAK2 activity. In a specific embodiment the disease is associated with JAK1 and/or JAK2 activity. In a more specific embodiment the disease is associated with JAK1 activity.

Other objects and advantages will become apparent to those skilled in the art from a consideration of the ensuing detailed description.

It will be appreciated that the compound of the invention may be metabolized to yield biologically active metabolites.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term ‘substituted’ is to be defined as set out below. It should be further understood that the terms ‘groups’ and ‘radicals’ can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

As used herein the term ‘JAK’ relates to the family of Janus kinases (JAKs) which are cytoplasmic tyrosine kinases that transduce cytokine signaling from membrane receptors to STAT transcription factors. Four JAK family members are described, JAK1, JAK2, JAK3 and TYK2 and the term JAK may refer to all the JAK family members collectively or one or more of the JAK family members as the context indicates.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Therapeutically effective amount’ means the amount of a compound that, when administered to a subject for treating a disease, is sufficient to effect such treatment for the disease. The ‘therapeutically effective amount’ can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, ‘treating’ or ‘treatment’ relates to slowing the progression of the disease.

As used herein the term ‘allergy’ refers to the group of conditions characterized by a hypersensitivity disorder of the immune system including, allergic airway disease (e.g. asthma, rhinitis), sinusitis, eczema and hives, as well as food allergies or allergies to insect venom.

As used herein the term ‘inflammatory condition(s)’ refers to the group of conditions including, rheumatoid arthritis, osteoarthritis, juvenile idiopathic arthritis, psoriasis, psoriatic arthritis, allergic airway disease (e.g. asthma, rhinitis), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. Particularly the term refers to rheumatoid arthritis, osteoarthritis, allergic airway disease (e.g. asthma) and inflammatory bowel diseases.

As used herein the term ‘autoimmune disease(s)’ refers to the group of diseases including obstructive airways disease, including conditions such as COPD, asthma (e.g intrinsic asthma, extrinsic asthma, dust asthma, infantily asthma) particularly chronic or inveterate asthma (for example late asthma and airway hyperreponsiveness), bronchitis, including bronchial asthma, systemic lupus erythematosus (SLE), cutaneous lupus erythrematosis, lupus nephritis, dermatomyositis, Sjogren's syndrome, multiple sclerosis, psoriasis, dry eye disease, type I diabetes mellitus and complications associated therewith, atopic eczema (atopic dermatitis), contact dermatitis and further eczematous dermatitis, inflammatory bowel disease (e.g. Crohn's disease and ulcerative colitis), atherosclerosis and amyotrophic lateral sclerosis. Particularly the term refers to COPD, asthma, systemic lupus erythematosis, type I diabetes mellitus and inflammatory bowel disease.

As used herein the term ‘proliferative disease(s)’ refers to conditions such as cancer (e.g. uterine leiomyosarcoma or prostate cancer), myeloproliferative disorders (e.g. polycythemia vera, essential thrombocytosis and myelofibrosis), leukemia (e.g. acute myeloid leukaemia, acute and chronic lymphoblastic leukemia), multiple myeloma, psoriasis, restenosis, scleroderma or fibrosis. In particular the term refers to cancer, leukemia, multiple myeloma and psoriasis.

As used herein, the term ‘cancer’ refers to a malignant or benign growth of cells in skin or in body organs, for example but without limitation, breast, prostate, lung, kidney, pancreas, stomach or bowel. A cancer tends to infiltrate into adjacent tissue and spread (metastasise) to distant organs, for example to bone, liver, lung or the brain. As used herein the term cancer includes both metastatic rumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, glioblastoma, primary liver cancer, ovarian cancer, prostate cancer and uterine leiomyosarcoma.

As used herein the term ‘leukemia’ refers to neoplastic diseases of the blood and blood forming organs. Such diseases can cause bone marrow and immune system dysfunction, which renders the host highly susceptible to infection and bleeding. In particular the term leukemia refers to acute myeloid leukaemia (AML), and acute lymphoblastic leukemia (ALL) and chronic lymphoblastic leukaemia (CLL).

As used herein the term ‘transplantation rejection’ refers to the acute or chronic rejection of cells, tissue or solid organ allo- or xenografts of e.g. pancreatic islets, stem cells, bone marrow, skin, muscle, corneal tissue, neuronal tissue, heart, lung, combined heart-lung, kidney, liver, bowel, pancreas, trachea or oesophagus, or graft-versus-host diseases.

As used herein the term ‘diseases involving impairment of cartilage turnover’ includes conditions such as osteoarthritis, psoriatic arthritis, juvenile rheumatoid arthritis, gouty arthritis, septic or infectious arthritis, reactive arthritis, reflex sympathetic dystrophy, algodystrophy, Tietze syndrome or costal chondritis, fibromyalgia, osteochondritis, neurogenic or neuropathic arthritis, arthropathy, endemic forms of arthritis like osteoarthritis deformans endemica, Mseleni disease and Handigodu disease; degeneration resulting from fibromyalgia, systemic lupus erythematosus, scleroderma and ankylosing spondylitis.

As used herein the term ‘congenital cartilage malformation(s)’ includes conditions such as hereditary chondrolysis, chondrodysplasias and pseudochondrodysplasias, in particular, but without limitation, microtia, anotia, metaphyseal chondrodysplasia, and related disorders.

As used herein the term ‘disease(s) associated with hypersecretion of IL6’ includes conditions such as Castleman's disease, multiple myeloma, psoriasis, Kaposi's sarcoma and/or mesangial proliferative glomerulonephritis.

As used herein the term ‘disease(s) associated with hypersecretion of interferons includes conditions such as systemic and cutaneous lupus erythematosis, lupus nephritis, dermatomyositis, Sjogren's syndrome, psoriasis, rheumatoid arthritis.

‘Compound(s) of the invention’, and equivalent expressions, are meant to embrace the compound of the Formula as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

When ranges are referred to herein, for example but without limitation, C₁₋₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particularly useful prodrugs. In some cases it is desirable to prepare double ester type prodrugs such as (acyloxy)alkyl esters or ((alkoxycarbonyl)oxy)alkylesters. Particular such prodrugs are the C₁₋₈ alkyl, C₂₋₈ alkenyl, C₆₋₁₀ optionally substituted aryl, and (C₆₋₁₀ aryl)-(C₁₋₄ alkyl) esters of the compounds of the invention.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of the invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compound

The present invention is based on the identification that the compound of the invention is an inhibitor of JAK and that they are useful for the treatment of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons. The present invention also provides methods for the production of the compound of the invention, pharmaceutical compositions comprising a compound of the invention and methods for treating allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons by administering the compound of the invention. In a specific embodiment the compound of the invention is an inhibitor of JAK1 and/or JAK2. In a more particular embodiment, the compound of the invention inhibits JAK1 with a selectivity vs the other JAK family members of at least 10 fold. Such selectivity is expected to result in an improved safety profile, and decreased side-effects that may occur via off-target activity.

Accordingly, in a first aspect the present invention provides, the compound of the invention according to Formula (I):

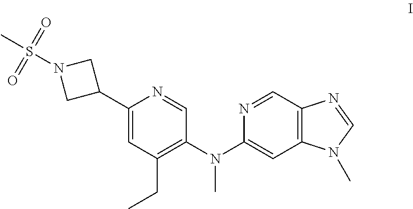 [SMILES: CCc1cc(C2CN(S(C)(=O)=O)C2)ncc1N(C)c1cc2c(cn1)ncn2C]

In one embodiment the compound of the invention is not an isotopic variant.

In one aspect the compound of the invention is present as the free base.

In one aspect the compound of the invention is a pharmaceutically acceptable salt.

In one aspect the compound of the invention is a solvate of the compound.

In one aspect the compound of the invention is a solvate of a pharmaceutically acceptable salt of a compound.

In certain aspects, the present invention provides prodrugs and derivatives of the compounds according to the formula above. Prodrugs are derivatives of the compound of the invention, which have metabolically cleavable groups and become by solvolysis or under physiological conditions the compound of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H. Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C₁ to C₈ alkyl, C₂-C₈ alkenyl, aryl, C₇-C₁₂ substituted aryl, and C₇-C₁₂ arylalkyl esters of the compounds of the invention.

The compound of the invention is a novel inhibitor of JAK. In particular, the compound is a potent inhibitor of JAK1 and/or JAK2; however it may inhibit TYK2 and JAK3 with a lower potency.

### Pharmaceutical Compositions

When employed as a pharmaceutical, the compound of the invention is typically administered in the form of a pharmaceutical composition. Such compositions can be prepared in a manner well known in the pharmaceutical art and comprise at least one active compound. Generally, a compound of this invention is administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the invention can be administered by a variety of routes including oral, rectal, transdermal, subcutaneous, intra-articular, intravenous, intramuscular, and intranasal. Depending on the intended route of delivery, a compound of this invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

The compound of the invention may be admixed as a dry powder with a dry gelatin binder in an approximate 1:2 weight ratio. A minor amount of magnesium stearate may be added as a lubricant. The mixture may be formed into 240-270 mg tablets (80-90 mg of active amide compound per tablet) in a tablet press.

### Formulation 2—Capsules

The compound of the invention may be admixed as a dry powder with a starch diluent in an approximate 1:1 weight ratio. The mixture may be filled into 250 mg capsules (125 mg of active amide compound per capsule).

### Formulation 3—Liquid

The compound of the invention (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

The compound of the invention may be admixed as a dry powder with a dry gelatin binder in an approximate 1:2 weight ratio. A minor amount of magnesium stearate may be added as a lubricant. The mixture may be formed into 450-900 mg tablets (150-300 mg of active amide compound) in a tablet press.

### Formulation 5—Injection

The compound of the invention may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of the compound of the invention (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Methods of Treatment

The compound of the invention may be used as a therapeutic agent for the treatment of conditions in mammals that are causally related or attributable to aberrant activity of JAK. In particular, conditions related to aberrant activity of JAK1 and/or JAK2, and more particularly conditions related to aberrant activity of JAK1. Accordingly, the compound and pharmaceutical compositions of the invention find use as therapeutics for preventing and/or treating allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons in mammals including humans.

In one aspect, the present invention provides the compound of the invention, or a pharmaceutical composition comprising the compound of the invention for use as a medicament.

In another aspect, the present invention provides the compound of the invention, or a pharmaceutical composition comprising the compound of the invention for use in the manufacture of a medicament.

In yet another aspect, the present invention provides a method of treating a mammal having, or at risk of having a disease disclosed herein, said method comprising administering an effective condition-treating or condition-preventing amount of one or more of the pharmaceutical compositions or the compound of the invention herein described. In a particular aspect, the present invention provides a method of treating a mammal having, or at risk of having allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

In a method of treatment aspects, this invention provides methods of treatment and/or prophylaxis of a mammal susceptible to or afflicted with an allergic reaction, said method comprising administering an effective condition-treating or condition-preventing amount of one or more of the pharmaceutical compositions or the compound of the invention herein described. In a specific embodiment, the allergic reaction is selected from allergic airway disease, sinusitis, eczema and hives, food allergies and allergies to insect venom.

In another aspect the present invention provides the compound of the invention for use in the treatment, and/or prophylaxis of an allergic reaction. In a specific embodiment, the allergic reaction is selected from allergic airway disease, sinusitis, eczema and hives, food allergies and allergies to insect venom.

In yet another aspect, the present invention provides the compound of the invention, or a pharmaceutical composition comprising the compound of the invention for use in the manufacture of a medicament for the treatment, or prophylaxis of an allergic reaction. In a specific embodiment, the allergic reaction is selected from allergic airway disease, sinusitis, eczema and hives, food allergies and allergies to insect venom.

In additional method of treatment aspects, this invention provides methods of treatment and/or prophylaxis of a mammal susceptible to or afflicted with an inflammatory condition. The methods comprise administering an effective condition-treating or condition-preventing amount of one or more of the pharmaceutical compositions or the compound of the invention herein described. In a specific embodiment, the inflammatory condition is selected from rheumatoid arthritis, osteoarthritis, allergic airway disease (e.g. asthma) and inflammatory bowel diseases.

In another aspect the present invention provides the compound of the invention for use in the treatment, and/or prophylaxis of an inflammatory condition. In a specific embodiment, the inflammatory condition is selected from rheumatoid arthritis, osteoarthritis, allergic airway disease (e.g. asthma) and inflammatory bowel diseases.

In yet another aspect, the present invention provides the compound of the invention, or a pharmaceutical composition comprising the compound of the invention for use in the manufacture of a medicament for the treatment, and/or prophylaxis of an inflammatory condition. In a specific embodiment, the inflammatory condition is selected from rheumatoid arthritis, osteoarthritis, allergic airway disease (e.g. asthma) and inflammatory bowel diseases.

In additional method of treatment aspects, this invention provides methods of treatment and/or prophylaxis of a mammal susceptible to or afflicted with an autoimmune disease. The methods comprise administering an effective condition-treating or condition-preventing amount of one or more of the pharmaceutical compositions or compounds of the invention herein described. In a specific embodiment, the autoimmune disease is selected from COPD, asthma, systemic lupus erythematosis, type I diabetes mellitus and inflammatory bowel disease.

In another aspect the present invention provides the compound of the invention for use in the treatment, and/or prophylaxis of an autoimmune disease. In a specific embodiment, the autoimmune disease is selected from COPD, asthma, systemic lupus erythematosis, type I diabetes mellitus and inflammatory bowel disease. In a more specific embodiment, the autoimmune disease is systemic lupus erythematosis.

In yet another aspect, the present invention provides the compound of the invention, or a pharmaceutical composition comprising the compound of the invention for use in the manufacture of a medicament for the treatment, and/or prophylaxis of an autoimmune disease. In a specific embodiment, the autoimmune disease is selected from COPD, asthma, systemic lupus erythematosis, type I diabetes mellitus and inflammatory bowel disease.

In further method of treatment aspects, this invention provides methods of treatment and/or prophylaxis of a mammal susceptible to or afflicted with a proliferative disease, said methods comprising administering an effective condition-treating or condition-preventing amount of one or more of the pharmaceutical compositions or the compound of the invention herein described. In a specific embodiment, the proliferative disease is selected from cancer (e.g. solid tumors such as uterine leiomyosarcoma or prostate cancer), leukemia (e.g. AML, ALL or CLL), multiple myeloma and psoriasis. In a more particular embodiment, the proliferative disease is selected from lung and hepatic cancer. In a more particular embodiment, the proliferative disease is selected from non-small-cell lung carcinoma (NSCLC), and hepatocellular carcinoma (HCC).

In another aspect the present invention provides the compound of the invention for use in the treatment, and/or prophylaxis of a proliferative disease. In a specific embodiment, the proliferative disease is selected from cancer (e.g. solid tumors such as uterine leiomyosarcoma or prostate cancer), leukemia (e.g. AML, ALL or CLL), multiple myeloma and psoriasis. In a more particular embodiment, the proliferative disease is selected from lung and hepatic cancer. In a more particular embodiment, the proliferative disease is selected from non-small-cell lung carcinoma (NSCLC), and hepatocellular carcinoma (HCC).

In yet another aspect, the present invention provides the compound of the invention, or a pharmaceutical composition comprising the compound of the invention for use in the manufacture of a medicament for the treatment, and/or prophylaxis of a proliferative disease. In a specific embodiment, the proliferative disease is selected from cancer (e.g. solid tumors such as uterine leiomyosarcoma or prostate cancer), leukemia (e.g. AML, ALL or CLL), multiple myeloma and psoriasis. In a more particular embodiment, the proliferative disease is selected from lung and hepatic cancer. In a more particular embodiment, the proliferative disease is selected from non-small-cell lung carcinoma (NSCLC), and hepatocellular carcinoma (HCC).

In further method of treatment aspects, this invention provides methods of treatment and/or prophylaxis of a mammal susceptible to or afflicted with transplantation rejection, said methods comprising administering an effective condition-treating or condition-preventing amount of one or more of the pharmaceutical compositions or compound of the invention herein described. In a specific embodiment, the transplantation rejection is organ transplant rejection.

In another aspect the present invention provides the compound of the invention for use in the treatment, and/or prophylaxis of transplantation rejection. In a specific embodiment, the transplantation rejection is organ transplant rejection.

In yet another aspect, the present invention provides the compound of the invention, or a pharmaceutical composition comprising the compound of the invention for use in the manufacture of a medicament for the treatment and/or prophylaxis of transplantation rejection. In a specific embodiment, the transplantation rejection is organ transplant rejection.

In a method of treatment aspect, this invention provides a method of treatment, and/or prophylaxis in a mammal susceptible to or afflicted with diseases involving impairment of cartilage turnover, which method comprises administering a therapeutically effective amount of the compound of the invention, or one or more of the pharmaceutical compositions herein described.

In another aspect the present invention provides the compound of the invention for use in the treatment, and/or prophylaxis of diseases involving impairment of cartilage turnover.

In yet another aspect, the present invention provides the compound of the invention, or a pharmaceutical composition comprising the compound of the invention for use in the manufacture of a medicament for the treatment, and/or prophylaxis of diseases involving impairment of cartilage turnover.

The present invention also provides a method of treatment and/or prophylaxis of congenital cartilage malformations, which method comprises administering an effective amount of one or more of the pharmaceutical compositions or the compound of the invention herein described.

In another aspect the present invention provides the compound of the invention for use in the treatment, and/or prophylaxis of congenital cartilage malformations.

In yet another aspect, the present invention provides the compound of the invention, or a pharmaceutical composition comprising the compound of the invention for use in the manufacture of a medicament for the treatment, and/or prophylaxis of congenital cartilage malformations.

In further method of treatment aspects, this invention provides methods of treatment and/or prophylaxis of a mammal susceptible to or afflicted with diseases associated with hypersecretion of IL6, said methods comprising administering an effective condition-treating or condition-preventing amount of one or more of the pharmaceutical compositions or compound of the invention herein described. In a specific embodiment, the disease associated with hypersecretion of IL6 is selected from Castleman's disease and mesangial proliferative glomerulonephritis.

In another aspect the present invention provides the compound of the invention for use in the treatment, and/or prophylaxis of diseases associated with hypersecretion of IL6. In a specific embodiment, the disease associated with hypersecretion of IL6 is selected from Castleman's disease and mesangial proliferative glomerulonephritis.

In yet another aspect, the present invention provides the compound of the invention, or a pharmaceutical composition comprising the compound of the invention for use in the manufacture of a medicament for the treatment, and/or prophylaxis of diseases associated with hypersecretion of IL6. In a specific embodiment, the disease associated with hypersecretion of IL6 is selected from Castleman's disease and mesangial proliferative glomerulonephritis.

In further method of treatment aspects, this invention provides methods of treatment and/or prophylaxis of a mammal susceptible to or afflicted with diseases associated with hypersecretion of interferons, said methods comprising administering an effective condition-treating or condition-preventing amount of one or more of the pharmaceutical compositions or the compound of the invention herein described. In a specific embodiment, the disease associated with hypersecretion of interferons is selected from systemic and cutaneous lupus erythematosis, lupus nephritis, dermatomyositis, Sjogren's syndrome, psoriasis, and rheumatoid arthritis.

In another aspect the present invention provides the compound of the invention for use in the treatment, and/or prophylaxis of diseases associated with hypersecretion of interferons. In a specific embodiment, the disease associated with hypersecretion of interferons is selected from systemic and cutaneous lupus erythematosis, lupus nephritis, dermatomyositis, Sjogren's syndrome, psoriasis, and rheumatoid arthritis.

In yet another aspect, the present invention provides the compound of the invention, or a pharmaceutical composition comprising the compound of the invention for use in the manufacture of a medicament for the treatment, and/or prophylaxis of diseases associated with hypersecretion of interferons. In a specific embodiment, the disease associated with hypersecretion of interferons is selected from systemic and cutaneous lupus erythematosis, lupus nephritis, dermatomyositis, Sjogren's syndrome, psoriasis, and rheumatoid arthritis.

As a further aspect of the invention there is provided the compound of the invention for use as a pharmaceutical especially in the treatment and/or prophylaxis of the aforementioned conditions and diseases. Also provided herein is the use of the present compound in the manufacture of a medicament for the treatment and/or prophylaxis of one of the aforementioned conditions and diseases.

A particular regimen of the present method comprises the administration to a subject suffering from a disease involving inflammation, of an effective amount of the compound of the invention for a period of time sufficient to reduce the level of inflammation in the subject, and preferably terminate the processes responsible for said inflammation. A special embodiment of the method comprises administering of an effective amount of the compound of the invention to a subject patient suffering from or susceptible to the development of rheumatoid arthritis, for a period of time sufficient to reduce or prevent, respectively, inflammation in the joints of said patient, and preferably terminate, the processes responsible for said inflammation.

A further particular regimen of the present method comprises the administration to a subject suffering from a disease condition characterized by cartilage or joint degradation (e.g. rheumatoid arthritis and/or osteoarthritis) of an effective amount of the compound of the invention for a period of time sufficient to reduce and preferably terminate the self-perpetuating processes responsible for said degradation. A particular embodiment of the method comprises administering of an effective amount of the compound of the invention to a subject patient suffering from or susceptible to the development of osteoarthritis, for a period of time sufficient to reduce or prevent, respectively, cartilage degradation in the joints of said patient, and preferably terminate, the self-perpetuating processes responsible for said degradation. In a particular embodiment said compound may exhibit cartilage anabolic and/or anti-catabolic properties.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prophylaxis and/or treatment of long-term conditions, such as degenerative conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound of the invention, with particular doses each providing from about 0.1 to about 10 mg/kg and especially about 1 to about 5 mg/kg.

Transdermal doses are generally selected to provide similar or lower blood levels than are achieved using injection doses.

When used to prevent the onset of a condition, the compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compound of the invention can be administered as the sole active agent or it can be administered in combination with other therapeutic agents, including other compounds that demonstrate the same or a similar therapeutic activity and that are determined to safe and efficacious for such combined administration. In a specific embodiment, co-administration of two (or more) agents allows for significantly lower doses of each to be used, thereby reducing the side effects seen.

In one embodiment, the compound of the invention or a pharmaceutical composition comprising the compound of the invention is administered as a medicament. In a specific embodiment, said pharmaceutical composition additionally comprises a further active ingredient.

In one embodiment, the compound of the invention is co-administered with another therapeutic agent for the treatment and/or prophylaxis of a disease involving inflammation; particular agents include, but are not limited to, immunoregulatory agents e.g. azathioprine, corticosteroids (e.g. prednisolone or dexamethasone), cyclophosphamide, cyclosporin A, tacrolimus, Mycophenolate Mofetil, muromonab-CD3 (OKT3, e.g. Orthocolone®), ATG, aspirin, acetaminophen, ibuprofen, naproxen, and piroxicam.

In one embodiment, the compound of the invention is co-administered with another therapeutic agent for the treatment and/or prophylaxis of arthritis (e.g. rheumatoid arthritis); particular agents include but are not limited to analgesics, non-steroidal anti-inflammatory drugs (NSAIDS), steroids, synthetic DMARDS (for example but without limitation methotrexate, leflunomide, sulfasalazine, auranofin, sodium aurothiomalate, penicillamine, chloroquine, hydroxychloroquine, azathioprine, and ciclosporin), and biological DMARDS (for example but without limitation Infliximab, Etanercept, Adalimumab, Rituximab, and Abatacept).

In one embodiment, the compound of the invention is co-administered with another therapeutic agent for the treatment and/or prophylaxis of proliferative disorders; particular agents include but are not limited to: methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), VEGF inhibitors (e.g. Avastin™), proteasome inhibitors (e.g. Velcade™), Glivec® and hsp90 inhibitors (e.g. 17-AAG). Additionally, the compound of the invention may be administered in combination with other therapies including, but not limited to, radiotherapy or surgery. In a specific embodiment the proliferative disorder is selected from cancer, myeloproliferative disease or leukaemia.

In one embodiment, the compound of the invention is co-administered with another therapeutic agent for the treatment and/or prophylaxis of autoimmune diseases, particular agents include but are not limited to: glucocorticoids, cytostatic agents (e.g. purine analogs), alkylating agents, (e.g nitrogen mustards (cyclophosphamide), nitrosoureas, platinum compounds, and others), antimetabolites (e.g. methotrexate, azathioprine and mercaptopurine), cytotoxic antibiotics (e.g. dactinomycin anthracyclines, mitomycin C, bleomycin, and mithramycin), antibodies (e.g. anti-CD20, anti-CD25 or anti-CD3 (OTK3) monoclonal antibodies, Atgam® and Thymoglobuline®), cyclosporin, tacrolimus, rapamycin (sirolimus), interferons (e.g. IFN-β), TNF binding proteins (e.g. infliximab (Remicade™) etanercept (Enbrel™), or adalimumab (Humira™)), mycophenolate, Fingolimod and Myriocin.

In one embodiment, the compound of the invention is co-administered with another therapeutic agent for the treatment and/or prophylaxis of transplantation rejection, particular agents include but are not limited to: calcineurin inhibitors (e.g. cyclosporin or tacrolimus (FK506)), mTOR inhibitors (e.g. sirolimus, everolimus), anti-proliferatives (e.g. azathioprine, mycophenolic acid), corticosteroids (e.g. prednisolone, hydrocortisone), Antibodies (e.g. monoclonal anti-IL-2Rα receptor antibodies, basiliximab, daclizumab), polyclonal anti-T-cell antibodies (e.g. anti-thymocyte globulin (ATG), anti-lymphocyte globulin (ALG)).

In one embodiment, the compound of the invention is co-administered with another therapeutic agent for the treatment and/or prophylaxis of asthma and/or rhinitis and/or COPD, particular agents include but are not limited to: beta2-adrenoceptor agonists (e.g. salbutamol, levalbuterol, terbutaline and bitolterol), epinephrine (inhaled or tablets), anticholinergics (e.g. ipratropium bromide), glucocorticoids (oral or inhaled) Long-acting 132-agonists (e.g. salmeterol, formoterol, bambuterol, and sustained-release oral albuterol), combinations of inhaled steroids and long-acting bronchodilators (e.g. fluticasone/salmeterol, budesonide/formoterol), leukotriene antagonists and synthesis inhibitors (e.g. montelukast, zafirlukast and zileuton), inhibitors of mediator release (e.g. cromoglycate and ketotifen), biological regulators of IgE response (e.g. omalizumab), antihistamines (e.g. ceterizine, cinnarizine, fexofenadine) and vasoconstrictors (e.g. oxymethazoline, xylomethazoline, nafazoline and tramazoline).

Additionally, the compound of the invention may be administered in combination with emergency therapies for asthma and/or COPD, such therapies include oxygen or heliox administration, nebulized salbutamol or terbutaline (optionally combined with an anticholinergic (e.g. ipratropium), systemic steroids (oral or intravenous, e.g. prednisone, prednisolone, methylprednisolone, dexamethasone, or hydrocortisone), intravenous salbutamol, non-specific beta-agonists, injected or inhaled (e.g. epinephrine, isoetharine, isoproterenol, metaproterenol), anticholinergics (IV or nebulized, e.g. glycopyrrolate, atropine, ipratropium), methylxanthines (theophylline, aminophylline, bamiphylline), inhalation anesthetics that have a bronchodilatory effect (e.g. isoflurane, halothane, enflurane), ketamine and intravenous magnesium sulfate.

In one embodiment, the compound of the invention is co-administered with another therapeutic agent for the treatment and/or prophylaxis of inflammatory bowel disease (IBD), particular agents include but are not limited to: glucocorticoids (e.g. prednisone, budesonide) synthetic disease modifying, immunomodulatory agents (e.g. methotrexate, leflunomide, sulfasalazine, mesalazine, azathioprine, 6-mercaptopurine and ciclosporin) and biological disease modifying, immunomodulatory agents (infliximab, adalimumab, rituximab, and abatacept).

In one embodiment, the compound of the invention is co-administered with another therapeutic agent for the treatment and/or prophylaxis of SLE, particular agents include but are not limited to: Disease-modifying antirheumatic drugs (DMARDs) such as antimalarials (e.g. plaquenil, hydroxychloroquine), immunosuppressants (e.g. methotrexate and azathioprine), cyclophosphamide and mycophenolic acid; immunosuppressive drugs and analgesics, such as nonsteroidal anti-inflammatory drugs, opiates (e.g. dextropropoxyphene and co-codamol), opioids (e.g. hydrocodone, oxycodone, MS Contin, or methadone) and the fentanyl duragesic transdermal patch.

In one embodiment, the compound of the invention is co-administered with another therapeutic agent for the treatment and/or prophylaxis of psoriasis, particular agents include but are not limited to: topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), Argan oiland retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™ Enbrel™, Humira™, Remicade™, Raptiva™ and ustekinumab (a IL-12 and IL-23 blocker). Additionally, the compound of the invention may be administered in combination with other therapies including, but not limited to phototherapy, or photochemotherapy (e.g. psoralen and ultraviolet A phototherapy (PUVA)).

In one embodiment, the compound of the invention is co-administered with another therapeutic agent for the treatment and/or prophylaxis of allergic reaction, particular agents include but are not limited to: antihistamines (e.g. cetirizine, diphenhydramine, fexofenadine, levocetirizine), glucocorticoids (e.g. prednisone, betamethasone, beclomethasone, dexamethasone), epinephrine, theophylline or anti-leukotrienes (e.g. montelukast or zafirlukast), anti-cholinergics and decongestants.

By co-administration is included any means of delivering two or more therapeutic agents to the patient as part of the same treatment regime, as will be apparent to the skilled person. Whilst the two or more agents may be administered simultaneously in a single formulation this is not essential. The agents may be administered in different formulations and at different times.

### General Synthetic Procedures

### General

The compound of the invention can be prepared from readily available starting materials using the following general methods and procedures. It will be appreciated that where typical or preferred process conditions (i.e. reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of a compound of the invention as defined hereinabove and the comparative examples. A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm) ¹H NMR spectra were recorded on a Bruker DPX 400 NMR spectrometer (400 MHz). Chemical shifts (δ) for ¹H NMR spectra are reported in parts per million (ppm) relative to tetramethylsilane (δ 0.00) or the appropriate residual solvent peak, i.e. CHCl₃ (δ 7.27), as internal reference. Multiplicities are given as singlet (s), doublet (d), triplet (t), quartet (q), multiplet (m) and broad (br). Coupling constants (J) are given in Hz. Electrospray MS spectra were obtained on a Micromass platform LC/MS spectrometer. Columns Used for LCMS analysis: Hichrom, Kromasil Eternity, 2.5 μm C18, 150×4.6 mm, Waters Xbridge 5 μm C18 (2), 250×4.6 mm (ref 86003117), Waters Xterra MS 5 μm C18, 100×4.6 mm (Plus guard cartridge) (ref 186000486), Gemini-NX 3 μm C18 100×3 0 mm (ref 00D-4453-Y0), Phenomenex Luna 5 μm C18 (2), 100×4.6 mm. (Plus guard cartridge) (ref 00D-4252-E0), Kinetix fused core 2.7 μm C18 100×4.6 mm (ref 00D-4462-E0), Supelco, Ascentis® Express C18 (ref 53829-U), or Hichrom Halo C18, 2.7 μm C18, 150×4.6 mm (ref 92814-702). LC-MS were recorded on a Waters Micromass ZQ coupled to a HPLC Waters 2795, equipped with a UV detector Waters 2996. LC were also run on a HPLC Agilent 1100 coupled to a UV detector Agilent G1315A. Preparative HPLC: Waters XBridge Prep C18 5 μm ODB 19 mm ID×100 mm L (Part No. 186002978). All the methods are using MeCN/H₂O gradients. H₂O contains either 0.1% TFA or 0.1% NH₃.

List of abbreviations used in the experimental section:

| Abbreviation | Definition |
|---|---|
| μL | microliter |
| 3- MOI | multiplicity of infection of 3 |
| Ad-Si RNA | Adenoviral encoded siRNA |
| APCI | atmospheric pressure chemical |
| | ionization |
| APMA | 4-aminophenylmercuric |
| | acetate |
| app t | Apparent triplet |
| aq | aqueous |
| AUC | Area Under the Curve |
| bd | Broad doublet |
| BINAP | 2,2′-bis(diphenylphosphino)- |
| | 1,1′-binaphthyl |
| Boc | tert-Butyloxy-carbonyl |
| Bs | broad singlet |
| Cat. | Catalytic amount |
| cDNA | copy deoxyribonucleic acid |
| Cpd | Compound |
| d | doublet |
| DCM | Dichloromethane |
| DMA | Dimethylacetamide |
| DMEM | Dulbecco′s Modified Eagle |
| | Medium |
| DMF | N,N-dimethylformamide |
| DMSO | Dimethylsulfoxide |
| dNTP | deoxyribonucleoside |
| | triphosphate |
| eq. | Equivalent |
| Et₂O | Diethyl ether |
| EtOAc | Ethyl acetate |
| EtOH | Ethanol |
| FBS | Fetal bovine serum |
| g | Gram |
| GAPDH | Glyceraldehyde phosphate |
| | dehydrogenase |
| h | Hour |
| hCAR | human cellular adenovirus |
| | receptor |
| HPLC | High pressure liquid |
| | chromatography |
| Int | Intermediate |
| iPrOH | Isopropanol |
| kg | kilogram |
| L | liter |
| m | multiplet |
| M | Moles per liter |
| MeCN | Acetonitrile |
| MeOH | Methanol |
| MeTHF | Methyl tetrahydrofuran |
| mg | milligram |
| min | minute |
| mL | millilitre |
| mmol | millimoles |
| MMP | Matrix Metallo Proteinase |
| MTBE | methyl t-butyl ether |
| MTT | 3-(4,5-dimethylthiazol-2-yl)- |
| | 2,5-diphenyltetrazolium |
| | bromide |
| MW | Molecular weight |
| nBuOH | n-Butanol |
| NMR | Nuclear Magnetic Resonance |
| Norit | Activated charcoal |
| Pd/C | Palladium on Carbon 10% |
| Pd₂(dba)₃ | Tris(dibenzylideneacetone) |
| | dipalladium(0) |
| PdCl₂dppf | [1,1′- |
| | Bis(diphenylphosphino)ferrocene] |
| | dichloropalladium(II) |
| PDX | Patient-Derived Xenografts |
| rel. vol | Relative volumes (e.g. volume |
| | in L, compared to solid weight |
| | in kg of limiting reagent) |
| rel. wgt | Relative weight (e.g. weight in |
| | kg, compared to sold weight in |
| | kg of limiting reagent) |
| RNA | Ribonucleic acid |
| Rt | retention time |
| s | singlet |
| SCID | severe combined |
| | immunodeficiency |
| SCX column | ion exchange sulfonic acid |
| | cross linked columns |
| t | triplet |
| TFA | Trifluoroacetic acid |
| THF | Tetrahydrofuran |
| ppm | part-per-million |
| q | quadruplet |
| TLC | Thin layer chromatography |
| X-Phos | 2-dicyclohexylphosphino- |
| | 2′,4′,6′-triisopropylbiphenyl |

### Synthetic Preparation of the Compound of the Invention

### General Synthetic Method

### Synthesis of intermediates

### Intermediate 1/Intermediate 2

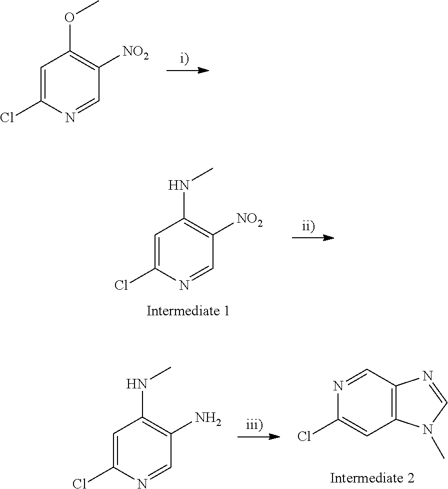

### Step (i): (2-Chloro-5-nitro-pyridin-4-yl)-methyl-amine (Intermediate 1)

To a solution of 2-chloro-4-methoxy-5-nitro-pyridine (0.026 mol) in dry THF (50 mL) at room temperature was added methyl amine (25 mL) (2M in THF). The mixture was allowed to stir for a further 2 h at room temperature. After completion of reaction as seen by TLC and LCMS, solvent was evaporated under reduced pressure to give 5 g of desired Intermediate 1.

¹H-NMR (400 MHz, DMSO-d₆): δ 2.95 (d, 3H), 7.01 (s, 1H), 8.57 (bs, 1H), 8.86, (s, 1H).

Mass (M+1): m/z 188.

### Step (ii): 6-Chloro-N-methyl-pyridine-3,4-diamine

To a stirred solution of Intermediate 1 (0.026 mol) in acetic acid (100 mL) was added iron powder (9 g, 0.16 mL) at 50° C. The reaction mixture was then heated at 80° C. for about 1 h when TLC showed the completion of reaction; it was cooled, filtered and washed with ethyl acetate (3×100 mL). Evaporation of organic layer gave residual mass, which was then neutralized with aq. NaHCO₃ solution and extracted with ethyl acetate (3×100 mL). Combined organic layers were washed with water (2×100 mL) dried over anhydrous sodium sulphate and concentrated under reduced pressure to give the desired compound.

¹H-NMR (400 MHz, DMSO-d₆): δ 2.74 (d, 3H), 4.66 (s, 2H), 6.25 (s, 1H), 7.36 (s, 1H).

Mass (M+1): m/z 158.

### Step (iii) 6-Chloro-1-methyl-1H-imidazo[4,5-c]pyridine: (intermediate 2)

To a stirred solution of 6-Chloro-N-methyl-pyridine-3,4-diamine (22 mmol) in trimethyl orthoformate (25 mL) was added formic acid (1 mL) and was heated at 100° C. for nearly 4 h when TLC showed the completion of reaction. The reaction was allowed to cool to room temperature and water (50 mL) was added and the mixture was extracted with ethyl acetate (4×50 mL), the combined organic layers were washed with aq. NaHCO₃ solution, dried over anhydrous sodium sulphate and concentration under reduced pressure gave the desired product Intermediate 2.

¹H-NMR (400 MHz, DMSO-d₆): δ 3.84 (s, 3H), 7.83 (s, 1H), 8.39 (s, 1H), 8.74 (s, 1H).

Mass (M+1): m/z 168.

### Compound 1: [4-Ethyl-6-(1-methanesulfonyl-azetidin-3-yl)-pyridin-3-yl]-methyl-(1-methyl-1H-imidazo[4,5-d]pyridin-6-yl)-amine

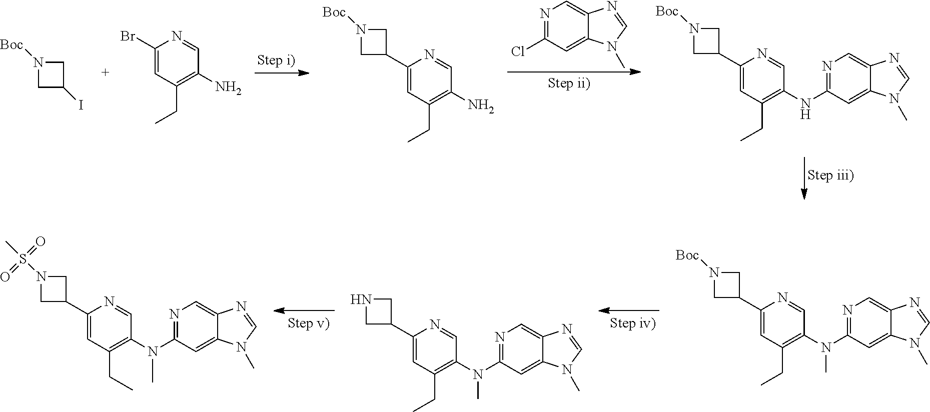

### Step i: 3-(5-Amino-4-ethyl-pyridin-2-yl)-azetidine-1-carboxylic acid tert-butyl ester

N-boc-azetidine-iodide (3 eq, 42 g) was dissolved in DMA (40 mL) and heated at 65° C. Under atmosphere of nitrogen, Rieke zinc (3.1 eq, 200 mL, 9.9 g) was added dropwise over 10 min and stirred for 20 min at 65° C. under nitrogen. In another flask, 6-bromo-4-ethyl-pyridin-3-ylamine (1 eq, 10 g), copper(I) iodide (0.01 eq, 100 mg) and Pd(dppf)Cl₂ (0.03 eq, 1.13 g) were dissolved in DMA (40 mL) and heated at 85° C. under atmosphere of nitrogen. The reaction mixture from the first flask was added via a cannula to the second flask over 10 min. The resulting reaction mixture was stirred at 85° C. for 5 min. The reaction was then quenched with aq. sat. NH₄Cl and extracted with EtOAc (3×200 mL). The combined organics was dried and concentrated under reduced pressure. The residue was purified by column chromatography (EtOAc//petroleum ether 40-60; 0:100 to 100:0) to give the desired product.

### Step ii: 3-[4-Ethyl-5-(1-methyl-1H-imidazo[4,5-c]pyridin-6-ylamino)-pyridin-2-yl]-azetidine-1-carboxylic acid tert-butyl ester

A mixture of 6-chloro-1-methyl-1H-imidazo[4,5-c]pyridine (Intermediate 2) (1.1 eq, 1.56 g), the aniline derivative obtained in step i (1.0 eq, 2.35 g) and Cs₂CO₃ (3.0 eq, 8.29 g) in dry dioxane (10 mL) was purged with nitrogen. Afterwards, Pd₂ dba₃ (0.1 eq, 0.78 g) and BINAP (0.2 eq, 1.06 g) were added, the reaction mixture was purged again with nitrogen and was stirred at 110° C. After 18 h, water was added and the mixture was extracted with EtOAc (3×). The organics were combined, dried and evaporated under reduced pressure to afford the desired product, which was used as such.

### Step iii: 3-{4-Ethyl-5-[methyl-(1-methyl-1H-imidazo[4,5-c]pyridin-6-yl)-amino]-pyridin-2-yl}-azetidine-1-carboxylic acid tert-butyl ester

NaH (60% dispersion in mineral oil, 2.6 eq, 1.76 g) was added to a dry THF solution (100 mL) of 3-[4-ethyl-5-(1-methyl-1H-imidazo[4,5-c]pyridin-6-ylamino)-pyridin-2-yl]-azetidine-1-carboxylic acid tert-butyl ester obtained in step II) above (1.0 eq, 6.92 g) at 0° C. After 30 min, iodomethane (2.0 eq, 2.1 mL) was added and the reaction was stirred at room temperature for 24 h. The reaction was quenched with cold water, the compound was extracted with DCM, dried and concentrated under vacuum. The compound was purified by silica flash chromatography (Interchim, Puriflash 450) (EtOAc/petroleum ether 40-60; 5:95 to 100:0) to afford the pure product.

### Step iv.: (6-Azetidin-3-yl-4-ethyl-pyridin-3-yl)-methyl-(1-methyl-1H-imidazo[4,5-c]pyridin-6-yl)-amine

The product of step iii) above (1.0 eq, 7.16 g) was added to a mixture TFA/DCM (1:1) (100 mL) and stirred at room temperature for 2 h. The resulting mixture was concentrated under vacuum. The residue was purified by a SCX column: the column was equilibrated with a solution of 5% AcOH in MeOH, the impurities were eluted with MeOH and the compound was eluted with a solution 2N NH₃ in MeOH to afford the desired product.

### Step v.: [4-Ethyl-6-(1-methanesulfonyl-azetidin-3-yl)-pyridin-3-yl]-methyl-(1-methyl-1H-imidazo[4,5-c]pyridin-6-yl)-amine

NEt₃ (1.1 eq, 3 mL) was added to a solution of the product of step iv) above (1.0 eq, 6.3 g) in DCM (290 mL) at 0° C. A solution of methylsulfonyl chloride 1M in DCM (1.1 eq, 21.5 mL) was added dropwise over 30 min and the resulting mixture was stirred at room temperature for 15 min. The mixture was diluted with DCM and washed with cold water. The organic layer was dried over Na₂SO₄, filtered and concentrated under vacuum. The residue was purified by silica flash chromatography (Interchim, Puriflash 450) (MeOH/EtOAc; 3:97 to 10:90) to afford the desired product.

¹H NMR δ (ppm) (CDCl₃): 8.69 (1H, d, ArH), 8.43 (1H, s, ArH), 7.67 (1H, s, ArH), 7.19 (1H, s, ArH), 6.01 (1H, d, ArH), 4.34-4.27 (4H, m, 2×CH₂), 4.02-3.94 (1H, m, CH), 3.64 (3H, s, CH₃), 3.44 (3H, s, CH₃), 3.03 (3H, s, CH₃), 2.51 (2H, q, CH₂), 1.16 (3H, t, CH₃).

Mass (M+1): m/z 401.0

### Alternative synthesis of Compound 1

### 1. Preparation of 6-Chloro-1-methyl-1H-imidazo[4,5-c]pyridine (Intermediate 1)

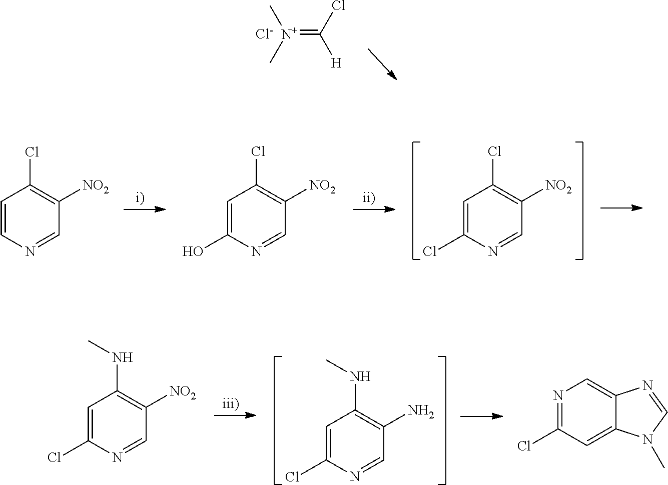

### Step 1): 4-Chloro-5-nitro-pyridin-2-ol

A reactor (A) is charged with potassium tert-butoxide (3.0 eq) and THF (12.5 rel. vol) and cooled to −35° C. Liquid ammonia (5 rel. vol) is then charged and the reactor contents cooled to −35 to −40° C.

In parallel, a second reactor (B) is charged with 4-chloro-3-nitropyridine (1.0 eq), THF (7.5 rel. vol) and cooled to −5° C. Tert-butylhydroperoxide in water (70%) (1.02 eq) is then added, and the contents of reactor (B) are transferred to reactor (A), while keeping the temperature between −35 to −40° C. After completion of the transfer, the reactor contents are post-stirred for 2 h between −35 to −40° C.

Afterwards, a solution of ammonium chloride (1.3 eq) in water (1.25 rel. vol) is added, while keeping the temperature between −35 to −40° C. Stirring is continued for 12 h, while allowing the reactor to warm up to ambient temperature. Subsequently, solvent is removed by vacuum distillation until an end volume of 15 rel. vol remained. Water (10 rel. vol) is added and again the solvent is removed (10 rel. vol) by vacuum distillation. The reactor contents are cooled to 20° C. and hydrochloric acid (30%) added until pH 5-6 is reached, while keeping the temperature below 25° C. The reactor contents are further cooled to 10° C., followed by stirring for 1 h causing the precipitation of a solid, which is separated by filtration. The cake is then washed twice with cold water (2 rel. vol) and dried.

### Step): (2-Chloro-5-nitro-pyridin-4-yl)-methyl-amine

A reactor (A) is charged with 4-chloro-3-nitropyridine (1.0 eq) and DCM (10 rel. vol) and cooled to 15° C. Subsequently charged with Vilsmeier reagent (CHCl=NMe₂)⁺Cl⁻ (1.35 eq) and stirring is continued for at least 6 h, while allowing the reactor contents to warm up to 20° C.

In parallel, a second reactor (B) is charged with water (5 rel. vol) and sodium bicarbonate (2.1 eq), followed by stirring for 5 min. Subsequently, the contents of reactor (A) are transferred to reactor (B) while keeping the reaction temperature at 20±5° C. The phases are separated and the aqueous layer discarded. The organic layer is concentrated by removal of solvent (5 rel. vol) by vacuum distillation until 6 rel. volumes remain. Isopropanol (5 rel. vol) is then added, followed by removal of solvent (6 rel. vol) until 5 rel. volumes remain in the reactor, which is then cooled to 0 to 5° C. Subsequently, methylamine in water (40%) (2.0 eq) is added, while keeping the reaction temperature below 15° C. Stirring is then continued for 30 to 60 min at 5° C. Water is added (6 vol), while keeping the temperature below 15° C. and the reactor contents cooled to 0 to 5° C., followed by stirring for 60 min at that temperature. The suspension is filtered and the cake washed twice with a cold mixture of isopropanol (0.8 rel. vol) and water (1.0 rel. vol), and dried to afford the desired compound.

### Step iii): 6-Chloro-1-methyl-1H-imidazo[4,5-c]pyridine

A reactor (A) is charged with 2-chloro-5-nitro-pyridin-4-yl)-methyl-amine (1.0 eq), Raney Nickel (0.15 wt-%) and a slurry of Norit activated charcoal (0.15 wt-%) in isopropanol (0.5 rel. vol). Additional isopropanol (9.5 rel. vol) is added and the temperature adjusted to 25 to 30° C. The reaction mixture is then hydrogenated for at least 6 h at 25 to 30° C., after which the reactor contents are transferred via a screening filter to a second reactor (B) (the reactor A is rinsed with isopropanol (2 rel. vol)). The solvent (6 rel. vol) is then removed by vacuum distillation between 80 to 90° C. until 7 relative volumes remain. Afterwards, triethyl orthoformate (4 eq) and formic acid (1 eq) are added and reactor contents heated to reflux (−83° C.). Stirring is continued for 30 min at reflux, after which additional formic acid (0.5 eq) and isopropanol (0.2 rel. vol) is added. Stirring at reflux is continued for 30 min, after which the reactor contents are cooled below 50° C. and triethylamine (1.0 eq) is added, followed by a rinse with isopropanol (0.2 rel. vol). Solvent (5 rel. vol) is removed at reflux until 7 relative volumes remain. Subsequently, the reactor contents are cooled to 10° C., during which the product crystallizes. The resulting suspension is filtered, washed with cold isopropanol (0.8 vol), and dried to afford the desired product (6-chloro-1-methyl-1H-imidazo[4,5-c]pyridine).

### 2. Preparation of 2-Chloro-4-ethyl-5-nitro-pyridine (Intermediate 3)

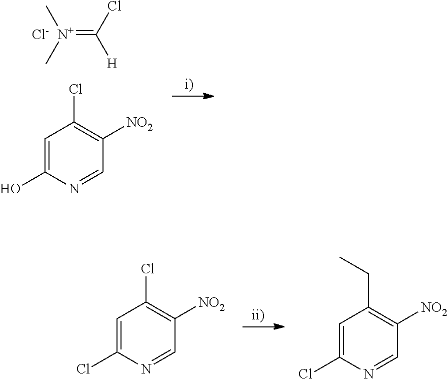

### Step i): 2,4-Dichloro-5-nitro-pyridine

A reactor (A) is charged with 4-chloro-5-nitro-pyridin-2-ol (1.0 eq) and DCM (8 rel. vol) and cooled to 15° C. Vilsmeier reagent (CHCl=NMe₂)⁺Cl⁻ (1.35 eq) is then added and stirring is continued for at least 6 h, while allowing the reactor contents to warm up to 20° C.

In parallel, a second reactor (B) is charged with water (5 rel. vol) and sodium bicarbonate (1.7 eq), followed by stirring for 5 min. Subsequently, the reactor (A) contents are transferred to reactor (B) via a screening filter (pre-packed with silica (1 rel. wgt), while keeping the reactor (B) temperature at 20±5° C. The phases are separated and the aqueous layer discarded. The organic layer is concentrated by removal of solvent (8 rel. vol) by vacuum distillation until 4.5 rel. volumes remain. Afterwards, isopropanol (4 rel. vol) is added, followed by removal of solvent (5 rel. vol) until 3 rel. volumes remain in the reactor, which is then cooled to 0 to 5° C. Subsequently, water (2.0 rel. vol) is added, while keeping the reaction temperature below 10° C. Stirring is then continued for 30 to 60 min at −10±5° C. The resulting suspension is filtered, and the filter cake is washed twice with a cold mixture of isopropanol (0.5 rel. vol) and water (0.5 rel. vol), and dried to afford the desired product (2,4-dichloro-5-nitro-pyridine).

### Step ii): 2-Chloro-4-ethyl-5-nitro-pyridine

A reactor (A) is charged with 2,4-dichloro-5-nitro-pyridine (1.0 eq), ethylboronic acid (1.1 eq), sodium carbonate (1.2 eq) and Pd(dppf)Cl₂ (0.04 eq).

Then toluene (7 rel. vol), water (1 rel. vol) and n-heptane (3 rel. vol) are charged. The reactor contents are purged with vacuum/nitrogen four times and heat to reflux (−85° C.). Stirring is continued for at least 16 h at reflux. After complete conversion, the reactor contents are cooled to 25±5° C. and filtered over a pre-coated filter with silica. The filter cake is rinsed once with MTBE (6 rel. vol) and the filtrate concentrated in vacuo. The residue is purified by column chromatography using MTBE/n-heptane as eluent and silica as stationary phase to afford the desired compound (2-Chloro-4-ethyl-5-nitro-pyridine, Intermediate 3).

### 3. Preparation of Compound 1: [4-Ethyl-6-(1-methanesulfonyl-azetidin-3-yl)-pyridin-3-yl]-methyl-(1-methyl-1H-imidazo[4,5-e]pyridin-6-yl)-amine

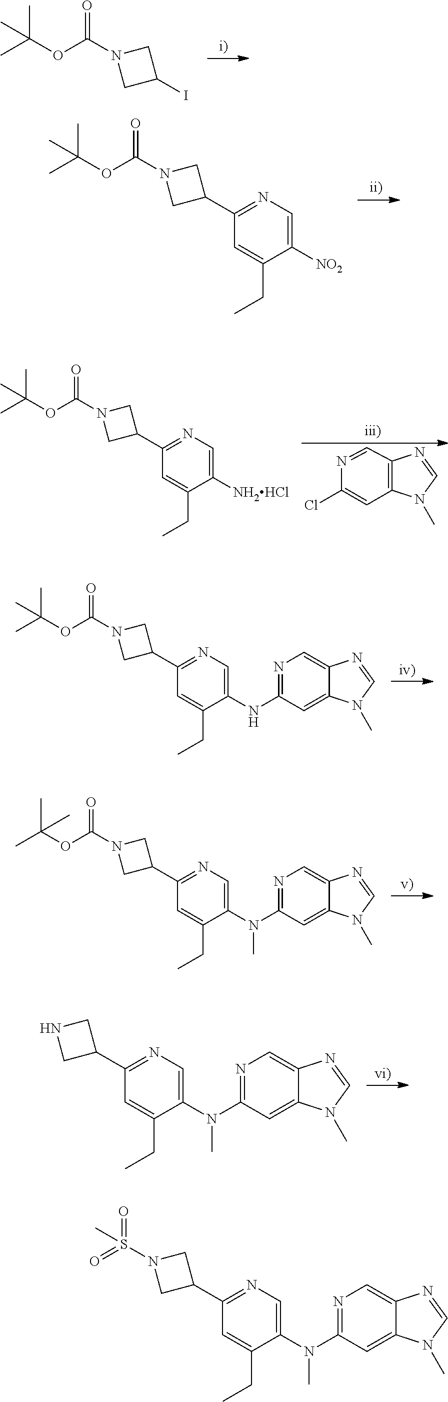

### Step i): 3-(5-nitro-4-ethyl-pyridin-2-yl)-azetidine-1-carboxylic acid tert-butyl ester

A reactor (A) is charged with Zn dust (2.47 eq), diatomous earth (dicalite) (0.033 rel wgt) and THF (4.33 rel. vol) and then heated to 25 to 30° C. An initial volume of a solution of N-Boc-3-iodo-azetidine (1.7 eq) in THF (13 vol) is added to reactor (A) and activated with iodine (0.05 eq). Once the reaction starts, the remainder of the N-Boc-3-iodo-azetidine solution is added, while keeping the temperature between 25 to 30° C. Upon completion of the addition, the mixture is post-stirred for 30 min between 25 to 30° C.

In parallel, a second reactor (B) is charged with Intermediate 3 (2-Chloro-4-ethyl-5-nitropyridine) (1.0 eq) and DMA (4.94 rel. vol). The resulting solution is purged with vacuum/nitrogen (3×) and warmed up to 50 to 55° C. Pd(dppf)Cl₂ (0.02 eq) and CuI (0.031 eq) are added, after which the solution of the organozincate in reactor (A) is transferred via a filter to reactor (B), while keeping the temperature between 50 to 55° C. The mixture is stirred for at least 1 h. The reactor contents are cooled to 35±5° C. and THF is distilled off between 35 and 45° C. until approximately 6 rel. vol remain. MTBE (5 rel. vol) is charged and a solution of ammonium chloride (2 rel. wght) in water (8.2 rel. vol). The mixture is post-stirred for 15 to 30 min, after which additional MTBE (15 rel. vol) is added and the phases are separated. The aqueous layer is extracted once with MTBE (5 vol) and afterwards discarded. The combined organic phases are extracted three times with water (2.5 rel. vol), after which the organic layer is filtered through silica (1 rel. wgt) and the filter cake rinsed once with MTBE (6.4 rel. vol). The filtrate is evaporated to dryness to afford the desired product which is used as such in the next step.

### Step ii): 3-(5-Amino-4-ethyl-pyridin-2-yl)-azetidine-1-carboxylic acid tert-butyl ester. hydrochloride salt

A reactor is charged with 3-(5-nitro-4-ethyl-pyridin-2-yl)-azetidine-1-carboxylic acid tert-butyl ester obtained in the previous step and THF (10 rel. vol). To the resulting solution, Raney Nickel (0.25 wgt) and Norit (0.25 wgt) are added and the reaction is stirred for at least 16 h under hydrogen (1 atm) at 20±5° C. The mixture is then cooled to 20° C. and the reactor content is filtered over dicalite. The filter cake is washed with THF (2 rel. vol). To the filtrate, 5-6M HCl in isopropanol (1.0 eq.) is added, while keeping the temperature below 20° C. After complete addition, the reactor contents are cooled to 0° C. and stirring continued for 1 hour. The solids are filtered and washed with THF (4 rel. vol), MTBE (2 rel. vol), and dried to afford the desired product.

### Step iii): 3-[4-Ethyl-5-(1-methyl-1H-imidazo[4,5-c]pyridin-6-ylamino)-pyridin-2-yl]-azetidine-1-carboxylic acid tert-butyl ester

A reactor is charged with 3-(5-amino-4-ethyl-pyridin-2-yl)-azetidine-1-carboxylic acid tert-butyl ester. hydrochloride salt (1.0 eq), Intermediate 1 (1.2 eq), Cs₂CO₃ (4 eq) and acetonitrile (10 vol). The reaction mixture is purged three times with vacuum/N₂, after which Pd₂(dba)₃ (0.05 eq) and X-Phos (0.2 eq) are added. The temperature is adjusted to allow stirring at reflux (82° C.) for at least 48 h. Afterwards, the solvent (2 rel. vol) is distilled and 2-MeTHF (2 rel. vol) added. The reaction mixture is cooled to 20° C. and the solids filtered. The filter cake is then rinsed twice with 2-MeTHF (4 rel. vol). The filtrate is concentrated in vacuo in order to remove acetonitrile (6 rel. vol). Subsequently the reaction mixture is stripped six times with 2-MeTHF by successive addition of 2-MeTHF (4 rel. vol) and removal of solvent (4 rel. vol). Finally, the solvent is evaporated in vacuo to yield the crude desired product which is used as such in the next step.

### Step iv): 3-{4-Ethyl-5-[methyl-(1-methyl-1H-imidazo[4,5-c]pyridin-6-yl)-amino]-pyridin-2-yl}-azetidine-1-carboxylic acid tert-butyl ester

A reactor is charged with NaH (2.0 eq) and THF (5 vol) and cooled to 0° C. Subsequently a solution of the product obtained in the previous step (1.0 eq) in THF (2.5 rel. vol) is added between 0 to 5° C. over 30 min. After rinsing with THF (0.5 vol), stirring is continued for 1.5 to 2.5 h at 0 to 5° C. until gas evolution ceases. To the reactor, methyliodide (2.0 eq) is added between 0 to 5° C., followed by a line rinse with THF (0.5 rel. vol). Stirring is continued for (at least) 4 h at 0 to 5° C., after which the reaction mixture is quenched with water (1.2 eq) and THF (0.5 rel. vol). THF (8 rel. vol) is removed by vacuum distillation, and additional 2-MeTHF (8 rel. vol) is added. Solvent is removed (8 rel. vol), then 2-MeTHF (8 rel. vol) and water (1 rel. vol) are added, the phases are separated, the aqueous layer is extracted twice with 2-MeTHF and discarded. The combined organic phases are washed twice with water (1 rel. vol) and concentrated in vacuo to yield the desired compound.

### Step v): (6-Azetidin-3-yl-4-ethyl-pyridin-3-yl)-methyl-(1-methyl-1H-imidazo[4,5-c]pyridin-6-yl)-amine

A reactor is charged with 3-{4-ethyl-5-[methyl-(1-methyl-1H-imidazo[4,5-c]pyridin-6-yl)-amino]-pyridin-2-yl}-azetidine-1-carboxylic acid tert-butyl ester (1.0 eq) obtained in the previous step and DCM (3 vol), and then cooled to −10° C. Slowly a solution of TFA (2.5 eq) in DCM (1.0 rel. vol) is added, while maintaining the temperature at −10° C. Afterwards, the reaction mixture is gradually warmed to 20° C. over 4 h and afterwards stirred overnight. Subsequently, it is cooled to 5° C., after which water (6 rel. vol) is added, while keeping the temperature 5° C. The phases are separated and the organic phase extracted twice with water (2 vol) before being discarded. The combined aqueous phases are extracted twice with DCM (2 rel. vol) and the organic phase discarded. Afterwards, DCM (4 rel. vol) is added, followed by dosing with 33% NaOH solution until pH>11, while keeping the temperature between 10 to 15° C. The phases are separated and the aqueous layer extracted four times with DCM (2 rel. vol) before being discarded. To the combined organic phases is added sodium sulfate (2 rel. wgt), followed by stirring for 2 h. After filtration of the solids and rinsing with DCM (4 rel. vol), the filtrate is evaporated in vacuo to afford the crude desired compound which is used as such in the next step.

### Step vi): Compound 1

A reactor is charged with the product obtained in the previous step (1.0 eq) and DCM (10 vol) and cooled to −15° C. Triethylamine (1.5 eq) is added, followed by cooling back to −15° C. Subsequently a solution of methanesulfonyl chloride (1.1 eq) in DCM (5 rel. vol) is dosed, while maintaining the temperature below −10° C. Afterwards, a solution of NaHCO₃ (0.25 wgt) in water (5 rel. vol) is dosed and the reaction mixture warmed to 20° C. After stirring for 15 min, the phases are separated and the aqueous layer discarded. The organic layer is extracted once with a solution of NaHCO₃ (0.25 wght) in water (5 rel. vol) and the aqueous layer discarded. The organic layer is treated with sodium sulfate (1.26 rel. wght), warmed to 20° C. and stirred for 1 hour. After filtration and rinsing of the filter cake twice with DCM (1.73 rel. vol), the filtrate is evaporated in vacuo to yield the desired crude Compound 1

Purification is accomplished by column chromatography using THF as eluent and silica as stationary phase.

Afterwards, the product obtained is further purified and crystallized according to following procedure:

The crude product (1.0 eq) is dissolved in EtOAc (7.44 rel. vol) and warmed to 40° C. Active carbon (0.15 wt-%) is charged and the suspension is stirred at 40° C. for 30 min. The suspension is then filtered and the solids washed twice with EtOAc (1 rel. vol). The filtrate is concentrated to dryness and re-dissolved in isopropyl acetate (2 rel. vol). The suspension is then heated to 40° C., after which water (0.04 rel. vol) is added between 30 to 40° C. and a minimal amount of seed material. Afterwards again water (0.02 rel. vol) is added between 30 to 40° C., after which stirring is continued for 2-4 h while allowing to cool to ambient temperature. The solids are filtered and the filter cake washed twice with water saturated isopropyl acetate (0.38 rel. vol) to afford the desired Compound 1.

### BIOLOGICAL EXAMPLES

### Example 1

### In-Vitro Assays

### 1.1 JAK1 Inhibition Assay

1.1.1 JAK1 Assay polyGT Substrate

Recombinant human JAK1 catalytic domain (amino acids 850-1154; catalog number 08-144) was purchased from Carna Biosciences. 10 ng of JAK1 is incubated with 12.5 μg polyGT substrate (Sigma catalog number P0275) in kinase reaction buffer (15 mM Tris-HCl pH 7.5, 1 mM DTT, 0.01% Tween-20, 10 mM MgCl₂, 2 μM non-radioactive ATP, 0.25 μCi³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 45 min at 30° C., reactions are stopped by adding of 25 μL/well of 150 mM phosphoric acid. All of the terminated kinase reaction is transferred to prewashed (75 mM phosphoric acid) 96 well filter plates (Perkin Elmer catalog number 6005177) using a cell harvester (Perkin Elmer). Plates are washed 6 times with 300 μL per well of a 75 mM phosphoric acid solution and the bottom of the plates is sealed. 40 μL/well of Microscint-20 is added, the top of the plates is sealed and readout is performed using the Topcount (Perkin Elmer). Kinase activity is calculated by subtracting counts per min (cpm) obtained in the presence of a positive control inhibitor (10 μM staurosporine) from cpm obtained in the presence of vehicle. The ability of a test compound to inhibit this activity is determined as:

Percentage inhibition=((cpm determined for sample with test compound present−cpm determined for sample with positive control inhibitor) divided by (cpm determined in the presence of vehicle−cpm determined for sample with positive control inhibitor))*100.

Dose dilution series are prepared for the compounds enabling the testing of dose-response effects in the JAK1 assay and the calculation of the IC₅₀ for each compound. Each compound is routinely tested at concentration of 20 μM followed by a ⅓ serial dilution, 8 points (20 μM-6.67 μM-2.22 μM-740 nM-247 nM-82 nM-27 nM-9 nM) in a final concentration of 1% DMSO. When potency of compound series increased, more dilutions are prepared and/or the top concentration was lowered (e.g. 5 μM, 1 μM).

### 1.1.2 JAK1 Ulight-JAK1 Peptide Assay

Recombinant human JAK1 (catalytic domain, amino acids 866-1154; catalog number PV4774) was purchased from Invitrogen. 1 ng of JAK1 was incubated with 20 nM Ulight-JAK1(tyr1023) peptide (Perkin Elmer catalog number TRF0121) in kinase reaction buffer (25 mM MOPS pH6.8, 0.01% Brij-35, 5 mM MgCl₂, 2 mM DTT, 7 μM ATP) with or without 4 μL containing test compound or vehicle (DMSO, 1% final concentration), in a total volume of 20 μL, in a white 384 Opti plate (Perkin Elmer, catalog number 6007290). After 60 min at room temperature, reactions were stopped by adding 20 μL/well of detection mixture (1× detection buffer (Perkin Elmer, catalog number CR97-100C), 0.5 nM Europium-anti-phosphotyrosine (PT66) (Perkin Elmer, catalog number AD0068), 10 mM EDTA). Readout is performed using the Envision with excitation at 320 nm and measuring emission at 615 nm (Perkin Elmer). Kinase activity was calculated by subtracting relative fluorescence units (RFU) obtained in the presence of a positive control inhibitor (10 μM staurosporine) from RFU obtained in the presence of vehicle. The ability of a test compound to inhibit this activity was determined as:

Percentage inhibition=((RFU determined for sample with test compound present−RFU determined for sample with positive control inhibitor) divided by (RFU determined in the presence of vehicle−RFU determined for sample with positive control inhibitor))*100.

Dose dilution series were prepared for the compounds enabling the testing of dose-response effects in the JAK1 assay and the calculation of the IC₅₀ for the compound. Each compound is routinely tested at concentration of 20 μM followed by a ⅕ serial dilution, 10 points in a final concentration of 1% DMSO. When potency of compound series increases, more dilutions are prepared and/or the top concentration are lowered (e.g. 5 μM, 1 μM). The data are expressed as the average IC₅₀ from the assays±standard error of the mean.

The compound of the invention has been tested for its activity against JAK1 using the assay described above and returned the following IC₅₀ values: 6.59, 12.87, 4.47, 10.15, 9.11, 5.42, 8.37, 9.43, 6.94 and 7.68 nM

### 1.1.3 JAK1 Ki Determination Assay

For the determination of Ki, different amounts of compound were mixed with the enzyme and the enzymatic reaction was followed as a function of ATP concentration. The Ki was determined by means of double reciprocal plotting of Km vs compound concentration (Lineweaver-Burk plot). 1 ng of JAK1 (Invitrogen, PV4774) is used in the assay. The substrate was 50 nM Ulight-JAK-1 (Tyr1023) Peptide (Perkin Elmer, TRF0121) The reaction is performed in 25 mM MOPS pH 6.8, 0.01%, 2 mM DTT, 5 mM MgCl₂ Brij-35 with varying concentrations of ATP and compound. Phosphorylated substrate was measured using an Eu-labeled anti-phosphotyrosine antibody PT66 (Perkin Elmer, AD0068) as described in 1.1.2. Readout was performed on the envision (Perkin Elmer) with excitation at 320 nm and emission followed at 615 nm and 665 nm.

The compound of the invention has been tested for its activity against JAK1 using the assay described above and returned the following Kᵢ value of 9.21 nM.

### 1.2 JAK2 Inhibition Assay

1.2.1 JAK2 Assay polyGT Substrate

Recombinant human JAK2 catalytic domain (amino acids 808-1132; catalog number PV4210) was purchased from Invitrogen. 0.025 mU of JAK2 is incubated with 2.5 μg polyGT substrate (Sigma catalog number P0275) in kinase reaction buffer (5 mM MOPS pH 7.5, 9 mM MgAc, 0.3 mM EDTA, 0.06% Brij and 0.6 mM DTT, 1 μM non-radioactive ATP, 0.25 μCi ³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 90 min at 30° C., reactions are stopped by adding of 25 μL/well of 150 mM phosphoric acid. All of the terminated kinase reaction is transferred to prewashed (75 mM phosphoric acid) 96 well filter plates (Perkin Elmer catalog number 6005177) using a cell harvester (Perkin Elmer). Plates are washed 6 times with 300 μL per well of a 75 mM phosphoric acid solution and the bottom of the plates is sealed. 40 μL/well of Microscint-20 is added, the top of the plates is sealed and readout is performed using the Topcount (Perkin Elmer). Kinase activity is calculated by subtracting counts per min (cpm) obtained in the presence of a positive control inhibitor (10 μM staurosporine) from cpm obtained in the presence of vehicle. The ability of a test compound to inhibit this activity is determined as:

Percentage inhibition=((cpm determined for sample with test compound present−cpm determined for sample with positive control inhibitor) divided by (cpm determined in the presence of vehicle−cpm determined for sample with positive control inhibitor))*100.

Dose dilution series are prepared for the compounds enabling the testing of dose-response effects in the JAK2 assay and the calculation of the IC₅₀ for each compound. Each compound is routinely tested at concentration of 20 μM followed by a ⅓ serial dilution, 8 points (20 μM-6.67 μM-2.22 μM-740 nM-247 nM-82 nM-27 nM-9 nM) in a final concentration of 1% DMSO. When potency of compound series increased, more dilutions are prepared and/or the top concentration is lowered (e.g. 5 μM, 1 μM).

### 1.2.2 JAK2 Ulight-JAK1 Peptide Assay

Recombinant human JAK2 (catalytic domain, amino acids 866-1154; catalog number PV4210) was purchased from Invitrogen. 0.0125 mU of JAK2 was incubated with 25 nM Ulight-JAK1(tyr1023) peptide (Perkin Elmer catalog number TRF0121) in kinase reaction buffer (25 mM HEPES pH7.0, 0.01% Triton X-100, 7.5 mM MgCl₂, 2 mM DTT, 7.5 μM ATP) with or without 4 μL containing test compound or vehicle (DMSO, 1% final concentration), in a total volume of 20 μL, in a white 384 Opti plate (Perkin Elmer, catalog number 6007290). After 60 min at room temperature, reactions were stopped by adding 20 μL/well of detection mixture (1× detection buffer (Perkin Elmer, catalog number CR97-100C), 0.5 nM Europium-anti-phosphotyrosine (PT66) (Perkin Elmer, catalog number AD0068), 10 mM EDTA). Readout is performed using the Envision with excitation at 320 nm and measuring emission at 615 nm (Perkin Elmer). Kinase activity was calculated by subtracting relative fluorescence units (RFU) obtained in the presence of a positive control inhibitor (10 μM staurosporine) from RFU obtained in the presence of vehicle. The ability of a test compound to inhibit this activity was determined as:

Percentage inhibition=((RFU determined for sample with test compound present−RFU determined for sample with positive control inhibitor) divided by (RFU determined in the presence of vehicle−RFU determined for sample with positive control inhibitor))*100.

Dose dilution series are prepared for compound enabling the testing of dose-response effects in the JAK2 assay and the calculation of the IC₅₀ for the compound. Each compound is routinely tested at concentration of 20 μM followed by a ⅕ serial dilution, 10 points in a final concentration of 1% DMSO. When potency of compound series increases, more dilutions are prepared and/or the top concentration are lowered (e.g. 5 μM, 1 μM). The data are expressed as the average IC₅₀ from the assays±standard error of the mean.

The compound of the invention has been tested for its activity against JAK2 using the assay described above and returned the following IC₅₀ values: 89.50, 140.4, 207.5, 106.6, 120.3, 80.42, 137.8, 173.1, 150.0, 16.2 and 177.5 nM.

### 1.2.3 JAK2 Ki Determination Assay

JAK2 (Invitrogen, PV4210) was used at a final concentration of 5 nM. The binding experiment was performed in 50 mM Hepes pH 7.5, 0.01% Brij-35, 10 mM MgCl₂, 1 mM EGTA using 25 nM kinase tracer 236 (Invitrogen, PV5592) and 2 nM Eu-anti-GST (Invitrogen, PV5594) with varying compound concentrations. Detection of tracer was performed according to the manufacturer's procedure.

The compound of the invention has been tested for its activity against JAK2 using the assay described above and returned the following Kᵢ value of 62.6 nM.

### 1.3 JAK3 Inhibition Assay

Recombinant human JAK3 catalytic domain (amino acids 781-1124; catalog number PV3855) was purchased from Invitrogen. 0.5 ng JAK3 protein was incubated with 2.5 μg polyGT substrate (Sigma catalog number P0275) in kinase reaction buffer (25 mM Tris pH 7.5, 0.5 mM EGTA, 10 mM MgCl₂, 2.5 mM DTT, 0.5 mM Na₃VO₄, 5 mM b-glycerolphosphate, 0.01% Triton X-100, 1 μM non-radioactive ATP, 0.25 μCi ³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 45 min at 30° C., reactions were stopped by adding 25 μL/well of 150 mM phosphoric acid. All of the terminated kinase reaction was transferred to prewashed (75 mM phosphoric acid) 96 well filter plates (Perkin Elmer catalog number

6005177) using a cell harvester (Perkin Elmer). Plates were washed 6 times with 300 μL per well of a 75 mM phosphoric acid solution and the bottom of the plates was sealed. 40 μL/well of Microscint-20 was added, the top of the plates was sealed and readout was performed using the Topcount (Perkin Elmer). Kinase activity was calculated by subtracting counts per min (cpm) obtained in the presence of a positive control inhibitor (10 μM staurosporine) from cpm obtained in the presence of vehicle. The ability of a test compound to inhibit this activity was determined as:

Percentage inhibition=((cpm determined for sample with test compound present−cpm determined for sample with positive control inhibitor) divided by (cpm determined in the presence of vehicle−cpm determined for sample with positive control inhibitor))*100.

Dose dilution series were prepared for the compounds enabling the testing of dose-response effects in the JAK3 assay and the calculation of the IC₅₀ for each compound. Each compound was routinely tested at concentration of 20 μM followed by a ⅕ serial dilution, 10 points in a final concentration of 1% DMSO. When potency of compound series increased, more dilutions were prepared and/or the top concentration was lowered (e.g. 5 μM, 1 μM).

The compound of the invention has been tested for its activity against JAK3 using the assay described above and returned the following IC₅₀ values: 1315, 523.3, 459.5, 344.9, 688.9, 685.3, 514.6, 324.2 and 536.9 nM.

### 1.3.1 JAK3 Ki Determination Assay

For the determination of Ki, different amounts of compound were mixed with the enzyme and the enzymatic reaction was followed as a function of ATP concentration. The Ki was determined by means of double reciprocal plotting of Km vs compound concentration (Lineweaver-Burk plot). JAK3 (Carna Biosciences, 09CBS-0625B) was used at a final concentration of 10 ng/mL. The substrate was Poly(Glu,Tyr)sodium salt (4:1), MW 20 000-50 000 (Sigma, P0275) The reaction was performed in 25 mM Tris pH 7.5, 0.01% Triton X-100, 0.5 mM EGTA, 2.5 mM DTT, 0.5 mM Na₃VO₄, 5 mM b-glycerolphosphate, 10 mM MgCl₂ with varying concentrations of ATP and compound and stopped by addition of 150 mM phosphoric acid. Measurement of incorporated phosphate into the substrate polyGT was done by loading the samples on a filter plate (using a harvester, Perkin Elmer) and subsequent washing. Incorporated ³³P in polyGT was measured in a Topcount scintillation counter after addition of scintillation liquid to the filter plates (Perkin Elmer).

The compound of the invention has been tested for its activity against JAK3 using the assay described above and returned the following Kᵢ values of 685 nM.

### 1.4 TYK2 Inhibition Assay

Recombinant human TYK2 catalytic domain (amino acids 871-1187; catalog number 08-147) was purchased from Carna biosciences. 5 ng of TYK2 was incubated with 12.5 μg polyGT substrate (Sigma catalog number P0275) in kinase reaction buffer (25 mM Hepes pH 7.2, 50 mM NaCl, 0.5 mM EDTA, 1 mM DTT, 5 mM MnCl₂, 10 mM MgCl₂, 0.1% Brij-35, 0.1 μM non-radioactive ATP, 0.125 μCi ³³P-gamma-ATP (GE Healthcare, catalog number AH9968) final concentrations) with or without 5 μL containing test compound or vehicle (DMSO, 1% final concentration), in a total volume of 25 μL, in a polypropylene 96-well plate (Greiner, V-bottom). After 90 min at 30° C., reactions were stopped by adding 25 μL/well of 150 mM phosphoric acid. All of the terminated kinase reaction was transferred to prewashed (75 mM phosphoric acid) 96 well filter plates (Perkin Elmer catalog number 6005177) using a cell harvester (Perkin Elmer). Plates were washed 6 times with 300 μL per well of a 75 mM phosphoric acid solution and the bottom of the plates was sealed. 40 μL/well of Microscint-20 was added, the top of the plates was sealed and readout was performed using the Topcount (Perkin Elmer). Kinase activity was calculated by subtracting counts per min (cpm) obtained in the presence of a positive control inhibitor (10 μM staurosporine) from cpm obtained in the presence of vehicle. The ability of a test compound to inhibit this activity was determined as:

Percentage inhibition=((cpm determined for sample with test compound present−cpm determined for sample with positive control inhibitor) divided by (cpm determined in the presence of vehicle−cpm determined for sample with positive control inhibitor))*100.

Dose dilution series were prepared for the compounds enabling the testing of dose-response effects in the TYK2 assay and the calculation of the IC₅₀ for each compound. Each compound was routinely tested at concentration of 20 μM followed by a ⅓ serial dilution, 8 points (20 μM-6.67 μM-2.22 μM-740 nM-247 nM-82 nM-27 nM-9 nM) in a final concentration of 1% DMSO. When potency of compound series increased, more dilutions were prepared and/or the top concentration was lowered (e.g. 5 μM, 1 μM).

The compound of the invention has been tested for its activity against TYK2 using the assay described above and returned the following IC₅₀ values: 844.3, 461.2, 470.4, 488.3, 759.3, 754.4, 1004, 501.4, and 687.8 nM.

### 1.4.1 TYK2 Ki Determination Assay

TYK2 (Carna Biosciences, 09CBS-0983D) was used at a final concentration of 5 nM. The binding experiment was performed in 50 mM Hepes pH 7.5, 0.01% Brij-35, 10 mM MgCl₂, 1 mM EGTA using 50 nM kinase tracer 236 (Invitrogen, PV5592) and 2 nM Eu-anti-GST (Invitrogen, PV5594) with varying compound concentrations. Detection of tracer was performed according to the manufacturers’ procedure.

The compound of the invention has been tested for its activity against TYK2 using the assay described above and returned the following Kᵢ value of 336 nM.

### Example 2

### Cellular Assays

### 2.1 JAK-STAT Signalling Assay

HeLa cells were maintained in Dulbecco's Modified Eagle's Medium (DMEM) containing 10% heat inactivated fetal calf serum, 100 U/mL penicillin and 100 μg/mL streptomycin. HeLa cells were used at 70% confluence for transfection. 20,000 cells in 87 μL cell culture medium were transiently transfected with 40 ng pSTAT1(2)-luciferase reporter (Panomics), 8 ng of LacZ reporter as internal control reporter and 52 ng of pBSK using 0.32 μL Jet-PEI (Polyplus) as transfection reagent per well in 96-well plate format. After overnight incubation at 37° C., 5% CO₂, transfection medium was removed. 81 μL of DMEM+1.5% heat inactivated fetal calf serum was added. 9 μL compound at 10× concentration was added for 60 min and then 10 μL of human OSM (Peprotech) at 33 ng/mL final concentration.

All compounds were tested in duplicate starting from 20 μM followed by a ⅓ serial dilution, 8 doses in total (20 μM-6.6 μM-2.2 μM-740 nM-250 nM-82 nM-27 nM-9 nM) in a final concentration of 0.2% DMSO.

After overnight incubation at 37° C., 5% CO₂ cells were lysed by adding 100 μL lysis buffer/well (PBS, 0.9 mM CaCl₂, 0.5 mM MgCl₂, 10% Trehalose, 0.05% Tergitol NP9, 0.3% BSA).

40 μL of cell lysate was used to read β-galactosidase activity by adding 180 μL β-Gal solution (30 μL ONPG 4 mg/mL+150 μL β-Galactosidase buffer (0.06 M Na₂HPO₄, 0.04 M NaH₂PO₄, 1 mM MgCl₂)) for 20 min. The reaction was stopped by addition of 50 μL Na₂CO₃ 1 M. Absorbance was read at 405 nm.

Luciferase activity was measured using 40 μL cell lysate plus 40 μL of Steadylite® as described by the manufacturer (Perkin Elmer), on the Envision (Perkin Elmer).

Omitting OSM was used as a positive control (100% inhibition). As negative control 0.5% DMSO (0% inhibition) was used. The positive and negative controls were used to calculate z′ and ‘percent inhibition’ (PIN) values.

Percentage inhibition=((fluorescence determined in the presence of vehicle−fluorescence determined for sample with test compound present) divided by (fluorescence determined in the presence of vehicle−fluorescence determined for sample without trigger))§100.

PIN values were plotted for compounds tested in dose-response and EC₅₀ values were derived.

The compound of the invention has been tested for its activity using the assay described above and returned the following IC₅₀ values: 1650 and 1661 nM.

### 2.2 OSM/IL-1β Signaling Assay

OSM and IL-1β are shown to synergistically upregulate MMP13 levels in the human chondrosarcoma cell line SW1353. The cells are seeded in 96 well plates at 15,000 cells/well in a volume of 120 μL DMEM (Invitrogen) containing 10% (v/v) FBS and 1% penicillin/streptomycin (InVitrogen) incubated at 37° C. 5% CO₂. Cells are preincubated with 15 μL of compound in M199 medium with 2% DMSO 1 h before triggering with 15 μL OSM and IL-1β to reach 25 ng/mL OSM and 1 ng/mL IL-1β, and MMP13 levels are measured in conditioned medium 48 h after triggering. MMP13 activity is measured using an antibody capture activity assay. For this purpose, 384 well plates (NUNC, 460518, MaxiSorb black) are coated with 35 μL of a 1.5 μg/mL anti-human MMP13 antibody (R&D Systems, MAB511) solution for 24 h at 4° C. After washing the wells 2 times with PBS+0.05% Tween, the remaining binding sites are blocked with 100 μL 5% non-fat dry milk (Santa Cruz, sc-2325, Blotto) in PBS for 24 h at 4° C. Next, the wells are washed twice with PBS+0.05% Tween and 35 μL of 1/10 dilution of culture supernatant containing MMP13 in 100-fold diluted blocking buffer is added and incubated for 4 h at room temperature. Next the wells are washed twice with PBS+0.05% Tween followed by MMP13 activation by addition of 35 μL of a 1.5 mM 4-Aminophenylmercuric acetate (APMA) (Sigma, A9563) solution and incubation at 37° C. for 1 hr. The wells are washed again with PBS+0.05% Tween and 35 μL MMP13 substrate (Biomol, P-126, OmniMMP fluorogenic substrate) is added. After incubation for 24 h at 37° C. fluorescence of the converted substrate is measured in a Perkin Elmer Wallac EnVision 2102 Multilabel Reader (wavelength excitation: 320 nm, wavelength emission: 405 nm).

Percentage inhibition=((fluorescence determined in the presence of vehicle−fluorescence determined for sample with test compound present) divided by (fluorescence determined in the presence of vehicle−fluorescence determined for sample without trigger))§100.

### 2.3 PBL Proliferation Assay

Human peripheral blood lymphocytes (PBL) are stimulated with IL-2 and proliferation is measured using a BrdU incorporation assay. The PBL are first stimulated for 72 h with PHA to induce IL-2 receptor, then they are fasted for 24 h to stop cell proliferation followed by IL-2 stimulation for another 72 h (including 24 hr BrdU labeling). Cells are preincubated with test compounds 1h before IL-2 addition. Cells are cultured in RPMI 1640 containing 10% (v/v) FBS.

### 2.4 Whole Blood Assay (WBA)

### 2.4.1 IFNα Stimulation Protocol

To predict the potency of the test compounds to inhibit JAK1 or JAK2-dependent signaling pathways in vivo, a physiologically relevant in vitro model was developed using human whole blood. In the WBA assay, blood, drawn from human volunteers who gave informed consent, is treated ex vivo with compound (1h) and subsequently stimulated either for 30 min with interferon α (IFNα, JAK1 dependent pathway) or for 2 h with granulocyte macrophage-colony stimulating factor (GM-CSF, JAK2 dependent pathway).

### 2.4.1.1 Phospho—STAT1 Assay

For IFNα stimulation, increase in phosphorylation of Signal Transducers and Activators of Transcription 1 (pSTAT1) by IFNα in white blood cell extracts is measured using a pSTAT1 ELISA assay. Phosphorylation of Signal Transducer and Activator of Transcription 1 (STAT1) after interferon alpha (IFNα) triggering is a JAK1-mediated event. The Phospho-STAT1 Assay, which is used to measure Phospho-STAT1 levels in cellular extracts, is developed to assess the ability of a compound to inhibit JAK1-dependent signaling pathways.

Whole human blood, drawn from human volunteers who gave informed consent, is ex vivo treated with compound (1h) and subsequently stimulated for 30 min with IFNα. The increase in phosphorylation of STAT1 by INFα in white blood cell extracts was measured using a phospho-STAT1 ELISA.

The ACK lysis buffer consisted of 0.15 M NH₄C1, 10 mM KHCO₃, 0.1 mM EDTA. The pH of the buffer was 7.3.

A 10× cell lysis buffer concentrate (part of the PathScan Phospho-STAT1 (Tyr701) sandwich ELISA kit from Cell Signaling) is diluted 10-fold in H₂O. Proteinase inhibitors were added to the buffer before use.

20 μg IFNα is dissolved in 40 μL H₂O to obtain a 500 μg/mL stock solution. The stock solution was stored at −20° C.

A 3-fold dilution series of the compound is prepared in DMSO (highest concentration: 10 mM). Subsequently, the compound is further diluted in medium (dilution factor dependent on desired final compound concentration).

2.4.1.1.1 Incubation of Blood with Compound and Stimulation with IFNα

Human blood is collected in heparinized tubes. The blood is divided in aliquots of 392 μL. Afterwards, 4 μL of compound dilution is added to each aliquot and the blood samples are incubated for 1 h at 37° C. The IFNα stock solution is diluted 1000-fold in RPMI medium to obtain a 500 ng/mL working solution. 4 μL of the 500 ng/mL work solution is added to the blood samples (final concentration IFNα: 5 ng/mL). The samples are incubated at 37° C. for 30 min.

### 2.4.1.1.2 Preparation of Cell Extracts

At the end of the stimulation period, 7.6 mL ACK buffer is added to the blood samples to lyse the red blood cells. The samples are mixed by inverting the tubes five times and the reaction is incubated on ice for 5 min. The lysis of the RBC should be evident during this incubation. The cells are pelleted by centrifugation at 300 g, 4° C. for 7 min and the supernatant is removed. 10 mL 1×PBS is added to each tube and the cell pellet is resuspended. The samples are centrifuged again for 7 min at 300 g, 4° C. The supernatant is removed and the pellet resuspended in 500 μL of 1×PBS. Then, the cell suspension is transferred to a clean 1.5 mL microcentrifuge tube. The cells are pelleted by centrifugation at 700 g for 5 min at 4° C. The supernatant is removed and the pellet was dissolved in 150 μL cell lysis buffer. The samples are incubated on ice for 15 min. After that, the samples are stored at −80° C. until further processing.

### 2.4.1.1.3 Measurement of STAT1 Phosphorylation by ELISA

The Pathscan Phospho-STAT1 (Tyr701) Sandwich ELISA kit from Cell Signaling (Cat. n°: #7234) is used to determine Phospho-STAT1 levels.

The cellular extracts are thawed on ice. The tubes are centrifuged for 5 min at 16,000 g, 4° C. and the cleared lysates are harvested. Meanwhile, the microwell strips from the kit are equilibrated to room temperature and wash buffer is prepared by diluting 20× wash buffer in H₂0, Samples are diluted 2-fold in sample diluent and 100 μL is added to the microwell strips. The strips are incubated overnight at 4° C.

The following day, the wells are washed 3 times with wash buffer. 100 μL of the detection antibody is added to the wells. The strips are incubated at 37° C. for 1 h. Then, the wells are washed 3 times with wash buffer again. 100 μL HRP-linked secondary antibody is added to each well and the samples are incubated at 37° C. After 30 min, the wells are washed 3 times again and 100 μL TMB substrate is added to all wells. When samples turned blue, 100 μL STOP solution is added to stop the reaction. Absorbance is measured at 450 nm.

### 2.4.1.2 Data Analysis

Inhibition of phosphoSTAT1 induction by IFNα in cell extracts is plotted against the compound concentration and IC₅₀ values are derived using Graphpad software. Data were retained if R² (coefficient of determination used in statistical models to measure the proportion of variability of the model and its predictive capacity. R² ranges from 0 (no correlation of the data: no predictive value) to 1 (full correlation: great predictive value) is larger than 0.8 and the hill slope is smaller than 3.

### 2.4.1.3 IL-8 ELISA

For GM-CSF stimulation, increase in interleukin-8 (IL-8) levels in plasma is measured using an IL-8 ELISA assay. Granulocyte macrophage-colony stimulating factor (GM-CSF)-induced interleukin 8 (IL-8) expression is a JAK2-mediated event. The IL-8 ELISA, which can be used to measure IL-8 levels in plasma samples, has been developed to assess the ability of a compound to inhibit JAK2-dependent signaling pathways.

Whole human blood, drawn from human volunteers who gave informed consent, is ex vivo treated with compound (1h) and subsequently stimulated for 2 h with GM-CSF. The increase in IL-8 levels in plasma is measured using an IL-8 ELISA assay.

10 μg GM-CSF is dissolved in 100 μL H₂O to obtain a 100 μg/mL stock solution. The stock solution is stored at −20° C.

A 3-fold dilution series of the test compound is prepared in DMSO (highest concentration: 10 mM). Subsequently, the compound is further diluted in medium (dilution factor dependent on desired final compound concentration).

2.4.1.3.1 Incubation of Blood with Compound and Stimulation with GM-CSF

Human blood is collected in heparinized tubes. The blood is divided in aliquots of 245 μL. Afterwards, 2.5 μL test compound dilution is added to each aliquot and the blood samples are incubated for 1 h at 37° C. The GM-CSF stock solution is diluted 100-fold in RPMI medium to obtain a 1 μg/mL work solution. 2.5 μL of the 1 μg/mL work solution is added to the blood samples (final concentration GM-CSF: 10 ng/mL). The samples are incubated at 37° C. for 2 h.

### 2.4.1.3.2 Preparation of Plasma Samples

The samples are centrifuged for 15 min at 1,000 g, 4° C. 100 μL of the plasma is harvested and stored at −80° C. until further use.

### 2.4.1.3.3 Measurement of IL-8 Levels by ELISA

The Human IL-8 Chemiluminescent Immunoassay kit from R&D Systems (Cat. n°: □8000B) is used to determine IL-8 levels.

Wash buffer is prepared by diluting 10× wash buffer in H₂O. Working glo reagent is prepared by adding 1 part Glo Reagent 1 to 2 parts Glo Reagent B 15 min to 4 h before use.

100 μL assay diluent RD1-86 is added to each well. After that, 50 μL of sample (plasma) is added. The ELISA plate is incubated for 2 h at room temperature, 500 rpm. All wells are washed 4 times with wash buffer and 200 μL IL-8 conjugate is added to each well. After incubation for 3 h at room temperature, the wells are washed 4 times with wash buffer and 100 μL working glo reagent is added to each well. The ELISA plate is incubated for 5 min at room temperature (protected from light). Luminescence is measured (0.5 s/well read time).

### 2.4.2 IL-6 Stimulation Protocol

In addition, a flow cytometry analysis was performed to establish JAK1 over JAK2 compound selectivity ex vivo using human whole blood. Therefore, blood was taken from human volunteers who gave informed consent. Blood was then equilibrated for 30 min at 37° C. under gentle rocking, then aliquoted in Eppendorf tubes. Compound was added at different concentrations and incubated at 37° C. for 30 min under gentle rocking and subsequently stimulated for 20 min at 37° C. under gentle rocking with interleukin 6 (IL-6) for JAK1-dependent pathway stimulation or GM-CSF for JAK2-dependent pathway stimulation. Phospho-STAT1 and phospho-STAT5 were then evaluated using FACS analysis.

### 2.4.2.1 Phospho-STAT1 Assays

For IL-6-stimulated increase of Signal Transducers and Activators of Transcription 1 (pSTAT1) phosphorylation in white blood cell, human whole blood, drawn from human volunteers who gave informed consent, was ex vivo treated with the compound for 30 min and subsequently stimulated for 20 min with IL-6. The increase in phosphorylation of STAT1 by IL-6 in lymphocytes was measured using anti phospho-STAT1 antibody by FACS.

The 5× Lyse/Fix buffer (BD PhosFlow, Cat. N°558049) was diluted 5-fold with distilled water and pre-warmed at 37° C. The remaining diluted Lyse/Fix buffer was discarded.

10 μg rhIL-6 (R&D Systems, Cat N°206-IL) was dissolved in 1 mL of PBS 0.1% BSA to obtain a 10 μg/mL stock solution. The stock solution was aliquoted and stored at −80° C.

A 3-fold dilution series of the compound was prepared in DMSO (10 mM stock solution). Control-treated samples received DMSO instead of compound. All samples were incubated with a 1% final DMSO concentration.

2.4.2.1.1 Incubation of Blood with Compound and Stimulation with IL-6

Human blood was collected in heparinized tubes. The blood was divided in aliquots of 148.5 μL. Then, 1.5 μL of the test compound dilution was added to each blood aliquot and the blood samples were incubated for 30 min at 37° C. under gentle rocking. IL-6 stock solution (1.5 μL) was d added to the blood samples (final concentration 10 ng/mL) and samples were incubated at 37° C. for 20 min under gentle rocking.

### 2.4.2.1.2 White Blood Cell Preparation and CD4 Labeling

At the end of the stimulation period, 3 mL of 1× pre-warmed Lyse/Fix buffer was immediately added to the blood samples, vortexed briefly and incubated for 15 min at 37° C. in a water bath in order to lyse red blood cells and fix leukocytes, then frozen at −80° C. until further use.

For the following steps, tubes were thawed at 37° C. for approximately 20 min and centrifuged for 5 min at 400×g at 4° C. The cell pellet was washed with 3 mL of cold 1×PBS, and after centrifugation the cell pellet was resuspended in 100 μL of PBS containing 3% BSA. FITC-conjugated anti-CD4 antibody or control FITC-conjugated isotype antibody were added and incubated for 20 min at room temperature, in the dark.

2.4.2.1.3 Cell Permeabilization and Labeling with Anti Phospho-STAT1 Antibody

After washing cells with 1×PBS, the cell pellet was resuspended in 100 μL of ice-cold 1×PBS and 900 μL ice-cold 100% MeOH was added. Cells were then incubated at 4° C. for 30 min for permeabilization.

Permeabilized cells were then washed with 1×PBS containing 3% BSA and finally resuspended in 80 μL of 1×PBX containing 3% BSA.

20 μL of PE mouse anti-STAT1 (pY701) or PE mouse IgG2aκ isotype control antibody (BD Biosciences, Cat. N°612564 and 559319, respectively) were added and mixed, then incubated for 30 min at 4° C., in the dark.

Cells are then washed once with 1×PBS and analyzed on a FACSCanto II flow cytometer (BD Biosciences).

### 2.4.2.1.4 Fluorescence Analysis on FACSCanto II

50,000 total events were counted and Phospho-STAT1 positive cells were measured after gating on CD4+ cells, in the lymphocyte gate. Data were analyzed using the FACSDiva software and the percentage inhibition of IL-6 stimulation calculated on the percentage of positive cells for phospho-STAT1 on CD4+ cells.

### 2.4.2.2 Phospho-STAT5 Assay

For GM-CSF-stimulated increase of Signal Transducers and Activators of Transcription 5 (pSTAT5) phosphorylation in white blood cell, human whole blood, drawn from human volunteers who gave informed consent, is ex vivo treated with compound for 30 min and subsequently stimulated for 20 min with GM-CSF. The increase in phosphorylation of STAT5 by GM-CSF in monocytes is measured using an anti phospho-STAT5 antibody by FACS.

The 5× Lyse/Fix buffer (BD PhosFlow, Cat. N°558049) is diluted 5-fold with distilled water and pre-warmed at 37° C. Remaining diluted Lyse/Fix buffer is discarded.

10 μg rhGM-CSF (AbCys S.A. Cat N°P300-03) is dissolved in 100 μL of PBS 0.1% BSA to obtain a 100 μg/mL stock solution. The stock solution is stored aliquoted at −80° C.

A 3-fold dilution series of the compound is prepared in DMSO (10 mM stock solution). Control-treated samples receive DMSO without the test compound. All samples are incubated with a 1% final DMSO concentration.

2.4.2.2.1 Incubation of Blood with Compound and Stimulation with GM-CSF

Human blood is collected in heparinized tubes. The blood is divided in aliquots of 148.5 μL. Then, 1.5 μL of compound dilution is added to each aliquot and the blood samples are incubated for 30 min at 37° C. under gentle rocking. GM-CSF stock solution (1.5 μL) is added to the blood samples (final concentration 20 pg/mL) and samples are incubated at 37° C. for 20 min under gentle rocking.

### 2.4.2.2.2 White Blood Cell Preparation and CD14 Labeling

At the end of the stimulation period, 3 mL of 1× pre-warmed Lyse/Fix buffer is immediately added to the blood samples, vortexed briefly and incubated for 15 min at 37° C. in a water bath in order to lyse red blood cells and fix leukocytes, then frozen at −80° C. until further use.

For the following steps, tubes are thawed at 37° C. for approximately 20 min and centrifuged for 5 min at 400×g at 4° C. The cell pellet is washed with 3 mL of cold 1×PBS, and after centrifugation the cell pellet is resuspended in 100 μL of PBS containing 3% BSA. FITC mouse anti-CD14 antibody (BD Biosciences, Cat. N°345784) or control FITC mouse IgG2bκ isotype antibody (BD Biosciences, Cat. N°555057) are added and incubated for 20 min at room temperature, in the dark.

2.4.2.2.3 Cell Permeabilization and Labeling with Anti Phospho-STAT5 Antibody

After washing cells with 1×PBS, the cell pellet is resuspended in 100 μL of ice-cold 1×PBS and 900 μL of ice-cold 100% MeOH is added. Cells are then incubated at 4° C. for 30 min for permeabilization.

Permeabilized cells are then washed with 1×PBS containing 3% BSA and finally resuspended in 80 μL of 1×PBX containing 3% BSA.

20 μL of PE mouse anti-STAT5 (pY694) or PE mouse IgG1κ isotype control antibody (BD Biosciences, Cat. N°612567 and 554680, respectively) are added, mixed then incubated for 30 min at 4° C., in the dark.

Cells are then washed once with 1×PBS and analyzed on a FACSCanto II flow cytometer (BD Biosciences).

### 2.4.2.2.4 Fluorescence Analysis on FACSCanto II

50,000 total events are counted and Phospho-STAT5 positive cells are measured after gating on CD14+ cells. Data are analyzed using the FACSDiva software and correspond to the percentage of inhibition of GM-CSF stimulation calculated on the percentage of positive cells for phosphor-STAT5 on CD 14+ cells.

### 2.4.2.2.5 Results

When submitted to these protocols, the compound according to Formula I returned a mean IC₅₀ of 498 nM on IL-6-induced STAT1 phosphorylation, on 9 different donors. On the GM-CSF-induced STAT5 phosphorylation, the mean IC₅₀ was evaluated to be over 22.400 μM in 8 different donors.

### 2.5 CTLL2 Vialibility Assay

The protocol describes the methods to analyse the activity of compounds on the ability to sustain the IL2-dependent viability of CTLL2 (ATCC TIB-214).

CTLL2 cells are cultured in RPMI1640 medium (life Technologies Cat n° 21875-034), with 10% fetal bovine serum (FBS, HiClone Cat n° SV30160.03), 1% penicillin/streptomycin and 10% T_STIM with ConA (BD Biosciences Cat n° 354115).

CTLL cells are seeded at 1000 cells per well of a white 384 well plate (Greiner, Cat n°781080) in 20 μl medium.

To the wells, 10 μL of diluted compound (or controls) is added. Negative control is a DMSO dilution, positive control at 10 μM. Final DMSO concentration is 0.1%.

The plates are incubated at 37° C. for 24 h and then the ATP content is measured using ATP-lite (Perkin Elmer, cat no 6016739). For this, 30 μL ATPlite solution is added to each well, and after 2 min shaking and another 8 min incubation at room temperature in the dark, bioluminescence is measured in a PerkinElmer Envision mutireader equipped for luminescence.

The compound of the invention has been tested for its activity using the assay described above and returned the following IC₅₀ values: 7390, and 3790 nM.

### 2.6 BA/F3 Viability Assay

The protocol describes the methods to analyse the activity of compounds on the ability to sustain the IL3-dependent viability of BA/F3 (ATCC CRL-12015; Collins et al, 1992).

BA/F3 cells are cultured in RPMI1640 medium (life Technologies Cat no 21875-034), with 10% fetal bovine serum (FBS, HiClone SV30160.03, 1% pen/strep and 10 ng/mL IL-3 (peprotech, no 213-13) BA/F3 cells are seeded at 1500 cells per well of a white 384 well plate (Greiner, 781080) in 20 μl medium. To the wells, 10 μL of diluted compound (or controls) is added. Negative control is a DMSO dilution, positive control is Tofacitinib at 10 μM. Final DMSO concentration is 0.1%.

The plates are incubated at 37° C. for 48 h and then the ATP content is measured using ATP-lite (Perkin Elmer, cat no 6016739). For this, 30 μL ATPlite solution is added to each well, and after 2 min shaking and another 8 min incubation at room temp in the dark, bioluminescence is measured in a PerkinElmer Envision mutireader equipped for luminescence.

The compound of the invention has been tested for its activity using the assay described above and returned the following IC₅₀ values: >11100, and 8022 nM.

### 2.7 JAK1, JAK2, and TYK2 Selectivity Cell Assays

### 2.7.1 Selective JAK1 Cell Assay, Activation of STAT1 by IFNα in PBMC

Pheripheral blood mononuclear cells (PBMC) are isolated from buffy coats under sterile conditions by density gradient centrifugation using LymphoPrep™ medium (Axis-Shield) followed by 3 subsequent wash steps in PBS without Ca++Mg++. PBMC are resuspended in plain RPMI 1640 medium containing 10% (v/v) heat inactivated FBS, 1% Penicillin/Streptomycin (100 U/mL Penicilium and 100 μg/mL Streptomycin) and further cultured in a humidified incubator at 37° C. 5% CO₂.

PBMC are seeded in 24 well plates at 5.0E06 cells/well in a volume of 200 μL RPMI 1640 (Invitrogen) containing 10% (v/v) FBS and 1% Penicillin/Streptomycin (Invitrogen).

PBMC are treated with test compound for 30 min at 37° C. 5% CO₂. 25 μL of 10× concentrated compound dilution is added to the medium. After 30 min of test compound/vehicle pre-treatment, PBMC are stimulated for 30 minutes at 37° C. 5% CO₂ with recombinant human IFNα (PeproTech) at final concentration of 100 ng/mL by addition of 25 μL (10× concentrated) cytokine trigger to obtain a final volume of 250 μL per well.

All compounds are tested in single starting from 20 μM followed by a ⅓ serial dilution, 8 doses in total (20 μM, 6.6 μM, 2.2 μM, 0.74 μM, 0.25 μM, 0.082 μM, 0.027 μM and 0.009 μM) in a final concentration of 0.2% DMSO.

After 30 minutes of cytokine stimulation, 250 μL of cell suspension is transferred to a 96-well V-bottom plate, centrifugated for 5 minutes at 1000 rpm to pellet cells, followed by removal of supernatant. The cell pellet is reconstituted in 100 μL 1× Lysis buffer supplemented with EDTA-free Protease Inhibitor Cocktail (Roche Applied Sciences, Product Number 11836170001) followed by sample freezing and storage at −80° C. 1× Lysis buffer is provided with the Phospho-STAT1 Elisa Kit and contains phosphatase inhibitors. Endogenous levels of phosphorylated STAT1 are quantified using a 96-well PathScan® Phospho-STAT1 (Tyr701) Sandwich ELISA Kit (Cell Signaling, Product Number #7234) according to manufacturer's instructions.

HRP activity (HRP is conjugated to the secondary antibody) is measured by addition of 100 μL of freshly prepared luminol substrate (BM Chemiluminescence ELISA Substrate (POD), Roche, Product Number 11582950001), incubation for 5 minutes at room temperature in the dark and measured in a Thermo Scientific Luminoskan Ascent Microplate Luminometer (integration time of 200 msec).

### 2.7.2 Selective JAK2 Cell Assay, Activation of STAT5 by GM-CSF in PBMC

Pheripheral blood mononuclear cells (PBMC) are isolated from buffy coats under sterile conditions by density gradient centrifugation using LymphoPrep™ medium (Axis-Shield) followed by 3 subsequent wash steps in PBS without Ca++Mg++. PBMC are resuspended in plain RPMI 1640 medium containing 10% (v/v) heat inactivated FBS, 1% Penicillin/Streptomycin (100 U/mL Penicilium and 100 μg/mL Streptomycin) and further cultured in a humidified incubator at 37° C. 5% CO₂.

PBMC are seeded in 24 well plates at 5.0E06 cells/well in a volume of 200 μL RPMI 1640 (Invitrogen) containing 10% (v/v) FBS and 1% Penicillin/Streptomycin (Invitrogen).

PBMC are treated with test compound by adding 25 μL of 10× concentrated compound dilution to the medium and incubated for 30 minutes at 37° C. 5% CO₂. Subsequently, PBMC are stimulated with recombinant human GM-CSF (PeproTech) at final concentration of 0.5 ng/mL by addition of 25 μL (10× concentrated) cytokine trigger per well to obtain a final volume of 250 μL. Cells are triggered for 30 minutes at 37° C. 5% CO₂.

All compounds are tested in single starting from 20 μM followed by a ⅓ serial dilution, 8 doses in total (20 μM, 6.6 μM, 2.2 μM, 0.74 μM, 0.25 μM, 0.082 μM, 0.027 μM and 0.009 μM) in a final concentration of 0.2% DMSO.

After 30 minutes of cytokine stimulation 250 μL of cell suspension is transferred to a 96-well V-bottom plate following centrifugation for 5 minutes at 1000 rpm to pellet cells. Cell supernatant is removed and pellet is reconstituted in 100 μL 1× Lysis buffer supplemented with EDTA-free Protease Inhibitor Cocktail (Roche Applied Sciences, Product Number 11836170001) followed by sample freezing and storage at −80° C. 1× Lysis buffer is provided with the Phospho-STAT5 Elisa Kit and contains phosphatase inhibitors. Endogenous levels of phosphorylated STAT5 are quantified using a 96-well PathScan® Phospho-STAT5 (Tyr694) Sandwich ELISA Kit (Cell Signaling, Product Number #7113) according to manufacturer's instructions.

HRP activity (HRP is conjugated to the secondary antibody) is measured by addition of 100 μL of freshly prepared luminol substrate (BM Chemiluminescence ELISA Substrate (POD), Roche, Product Number 11582950001), incubation for 5 minutes at room temperature in the dark and measured in a Thermo Scientific Luminoskan Ascent Microplate Luminometer (integration time of 200 msec).

### 2.7.3 Selective TYK2 Cell Assay, Activation of STAT4 by IL-12 in NK-92 Cells

NK-92 cells (human malignant non-Hodgkin's lymphoma, interleukin-2 (IL-2) dependent Natural Killer Cell line, ATCC #CRL-2407).

NK-92 cells are maintained in Minimum Essential Medium (MEM) Alpha medium w/o ribonucleosides and desoxyribonucleosides, 2 mM L-glutamine, 2.2 g/L sodium bicarbonate (Invitrogen, Product Number 22561-021) containing 0.2 mM myo-inositol, 0.1 mM 2-mercapto-ethanol, 0.1 mM folic acid, 12.5% heat inactivated horse serum (Invitrogen, Product Number 26050-088), 12.5% heat inactivated FBS, 1% Penicillin/Streptomycin (100 U/mL Penicilium and 100 μg/mL Streptomycin) and 10 ng/mL recombinant human IL-2 (R&D Systems). IL-2 is added freshly to the medium with each medium refreshment step. Cells are cultured in a humidified incubator at 37° C. 5% CO₂.

A subcultured fraction of NK-92 cells are washed once in plain medium without rhIL-2 and seeded in 24-well plates at 0.5E06 cells/well in a volume of 400 μL of plain Alpha MEM medium w/o rhIL-2 containing 0.2 mM myo-inositol, 0.1 mM 2-mercapto-ethanol, 0.1 mM folic acid, 12.5% heat inactivated horse serum (Invitrogen, Product Number 26050-088), 12.5% heat inactivated FBS, 1% Penicillin/Streptomycin (Invitrogen).

NK-92 cells are treated with test compounds for 30 minutes prior to rhIL-12 stimulation by adding 50 μL of 10× concentrated compound dilution and incubation at 37° C. 5% CO₂. After 30 minutes of compound/vehicle pre-treatment, cells are stimulated with recombinant human IL-12 (R&D Systems, Product Number 219-IL) at final concentration of 25 ng/mL by addition of 50 μL (10× concentrated) cytokine trigger to obtain a final volume of 500 μL per well. NK-92 cells are triggered with rhIL-12 for 30 minutes at 37° C. 5% CO₂.

All compounds are tested in single starting from 20 μM followed by a ⅓ serial dilution, 8 doses in total (20 μM, 6.6 μM, 2.2 μM, 0.74 μM, 0.25 μM, 0.082 μM, 0.027 μM and 0.009 μM) in a final concentration of 0.2% DMSO.

The levels of phospho-STAT4 in rhIL-12 stimulated NK-92 cells are quantified using a flow cytometric analysis on a Gallios™ flow cytometer (Beckman Coulter). After 30 minutes of cytokine stimulation the cells are fixed by adding 500 μL of pre-warmed BD Cytofix Fixation Buffer (BD Phosflow™, Product Number 554655) immediately to the wells (fix cells immediately in order to maintain phosphorylation state, rather than spinning down the cells, it is recommended to fix the cells by adding an equal volume of pre-warmed BD Cytofix Buffer to the cell suspension). Cells are incubated for 10 minutes at 37° C. The fixed cell fraction is resuspended (1 mL) and transferred to FACS tubes followed by a centrifugation step (300×g, 10 minutes) and removal of the supernatant. The cell pellet is mixed (vortex) and the cells are permeabilized by adding 1 mL of BD Phosflow Perm Buffer III (BD Phosflow™, Product Number 558050) followed by incubation on ice for 30 minutes. After the permeabilization step, the cells are washed twice with BD Pharmingen™ Stain Buffer (BD Pharmingen, Product Number 554656) with intermediate centrifugation at 300×g for 10 minutes and removal of the supernatant. The pellet (0.5E06 cells) is resuspended in 100 μL of BD Pharmingen™ Stain Buffer and stained by mixing 20 μL of PE Mouse Anti-STAT4 (pY693) to the cells (BD Phosflow™, PE Mouse Anti-STAT4 (pY693), Product Number 558249), then incubated for 30 minutes at room temperature in the dark. The stained cells are washed once with 2 mL of BD Pharmingen™ Stain Buffer and resuspended in 500 μL of BD Pharmingen™ Stain Buffer and analyzed on a Gallios™ flow cytometer (Beckman Coulter).

For all analyses, dead cells and debris are excluded by forward scatter (FSC) and side scatter (SSC). Changes in phosphorylation of STAT4 proteins following cytokine stimulation are approximated by calculating the X-median or X-mean fluorescence intensity (MFI) per cell on 100% of the gated fraction for all cytokine stimulated, test compound and unstimulated samples.

### 2.7.4 Results JAK1, JAK2 and TYK2 Assays:

Unstimulated samples (no trigger/vehicle (0.2% DMSO) are used as a positive control (100% inhibition). As a negative control (0% inhibition), the stimulated samples (trigger/vehicle (0.2% DMSO)) are used. The positive and negative controls are used to calculate Z′ and ‘percent inhibition (PIN)’ values.

Percentage inhibition is calculated from

$$Percentage inhibition = \frac{R C L U ( trigger / veh ) - R C L U ( test compound )}{R C L U ( trigger / veh ) - R C L U ( no trigger / veh )} \times 100$$

### Wherein

RCLU(trigger/veh): Relative Chemilumescent signal determined in presence of vehicle and trigger
RCLU(test compound): Relative Chemiluminescent signal determined in presence of test compounds)
RCLU(no trigger/veh): Relative Chemiluminescent signal determined in presence of vehicle without trigger.

In case the readout signal is expressed as X-mean values (flow cytometric analysis of pSTAT4 levels in cytokine stimulated NK-92 cells), the RCLU is replaced by X-mean value.

PIN values are plotted for compounds tested in dose-response and EC₅₀ values are derived using GraphPad Prism Software applying non-linear regression (sigmoidal) curve fitting.

### 2.8 JAK1 Mutations in Lung Cancer and Hepatocellular Carcinoma Cell Lines Assay.

### 2.8.1 JAK1 Mutation Induced Constitutive Signaling

Cancer cell lines with and without JAK1 mutations (Table I—Lung cancer cell lines) are cultured with or without serum for 4-6 h, stimulated or not with a cytokine cocktail (INFγ, IL2, IL4 and IL6) for 5, 10, 30 and 45 min. The phosphorylation of JAK1, STAT1, STAT3 and STAT5 are evaluated by immunoblot (Cell Signaling antibodies).

### 2.8.2 Targeting JAK1 Mutants Using JAK Inhibitors

### 2.8.2.1 JAK-STAT Pathway Phosphorylation:

Cancer cell lines with and without JAK1 mutations are cultured in the presence or absence of different concentrations of JAK inhibitors. Cells are analyzed at 24 and 48 h for effective JAK-STAT pathway inhibition by immunoblot.

TABLE I
Illustrative lung cancer cell lines

| | | | | | Present |
|---|---|---|---|---|---|
| | | | | Protein | in primary |
| Gene | Cell line | Tissue | Change | domain | tissue |
| JAK1 | NCIH1915 | Lung | I62V | FERM | — |
| JAK1 | SQ1 | Lung | N226S | FERM | — |
| JAK1 | HCC4006 | Lung | S383G | FERM | — |
| JAK1 | NCIH2066 | Lung | L423V | Interdomain | — |
| | | | | (FERM and | |
| | | | | SH2) | |
| JAK1 | NCIH1793 | Lung | H525Y | SH2 | — |
| JAK1 | HCC95 | Lung | N833S | Protein | Yes |
| | | | | kinase 1 | |
| JAK1 | VMRCLCD | Lung | E223* | — | — |
| JAK1 | NCIH1563 | Lung | Q161* | — | — |
| WT JAK1 | A549 | Lung | — | — | — |
| JAK1 −/− | U4C | Fibro- | — | — | — |
| | | sarcoma | | | |

*truncation

### 2.8.2.2 Cell Viability

2D-assay: Cancer cell lines with and without JAK1 mutations are cultured in the presence or absence of increasing concentrations of JAK inhibitors. After 48-72 h, cell viability is measured using the Cell Titer-Glo Luminescent cell viability assay (Promega) or MTT assay. Alternatively, cancer cell lines at different culture time points with a fix concentration of JAK inhibitor are analyzed for cell viability using the Cell Titer-Glo Luminescent cell viability assay (Promega) or MTT assay.

3D-assay: Cancer cell lines with and without JAK1 mutations are seeded in semi-solid agar medium. Formation of multi-cellular colonies is measured by determining cell viability using a fluorescent dye at different culture time points. Addition of potential inhibitors after cell seeding allows for the analyses of anti-tumorigenic effects.

### 2.8.3 Investigating Human JAK1 Mutations in Murine Ba/F3 Cells

(As illustrated in: Kan Z. et al. Genome Res 2013; 23:1422-33; Staerk J. et al. J Biol Chem 2005; 280:41893-41899; Zenatti P. P. et al. Nat. Gen. 2011; 43:932-41)

Construction of JAK1 expression vectors: Wild type and mutant human JAK1 sequences are cloned into retroviral vectors and clones verified by sequencing.

Retroviral infection of Ba/F3 cells: Ba/F3 cells are infected with retroviral supernatants produced in 293T cells.

Ba/F3 cells expressing human WT or mutated JAK1 are cultured with or without IL-3 for 4 h and phosphorylation of the JAK-STAT pathway evaluated by immunoblot.

The transforming potential of JAK1 mutations is assessed by measuring the ability of each mutation to induce autonomous growth when expressed in cytokine-dependent Ba/F3 cells. Cell growth is assessed in the absence of the cytokine IL-3.

Mutant JAK1 transduced Ba/F3 cell lines are assessed for their sensitivity to the JAK inhibitors by culturing them in the presence or absence of increasing concentrations of JAK inhibitors. After 48-72 h, cell viability is measured using the Cell Titer-Glo Luminescent cell viability assay (Promega) or MTT assay. Alternatively, cancer cell lines at different culture time points with a fix concentration of JAK inhibitor are analyzed for cell viability using the Cell Titer-Glo Luminescent cell viability assay (Promega) or MTT assay.

### 2.8.4 In Vivo Tumorigenic Potential of JAK1 Mutations

### 2.8.4.1 Xenograft Model:

Mutant JAK1 expressing cells are injected subcutaneously in CD1 nu/nu mice or Rag1−/− mice and evaluated for tumor progression. Subcutaneous tumor volume growth curves are established. The transplantability of primary tumors into secondary recipient animals is determined

### 2.8.4.2 PDX Model.

Patient-Derived Xenografts (PDXs) are based on the transfer of primary tumors (containing JAK1 mutations) directly from the patient into an immunodeficient mouse. To accomplish this, patient tumors must be obtained fresh from surgery, at which point they are mechanically or chemically digested, with a small portion saved as a primary stock and established in a NOD-SCID mouse. PDX models are maintained by passaging cells directly from mouse to mouse once the tumor burden becomes too high. Tumors can be engrafted heterotopically (implanting tumors into the subcutaneous flank of a mouse) or orthotopically (direct implantation to the mouse organ of choice).

The phosphorylation of JAK1, STAT1, STAT3 and STAT5 in primary and secondary tumors are evaluated by immunoblot.

### Example 3

### In Vivo Models

3.1 CIA model

### 3.1.1 Materials

Completed Freund's adjuvant (CFA) and incomplete Freund's adjuvant (IFA) were purchased from Difco. Bovine collagen type II (CII), lipopolysaccharide (LPS), and Enbrel was obtained from Chondrex (Isle d'Abeau, France); Sigma (P4252, L'Isle d'Abeau, France), Whyett (25 mg injectable syringe, France) Acros Organics (Palo Alto, Calif.), respectively. All other reagents used were of reagent grade and all solvents were of analytical grade.

### 3.1.2 Animals

Dark Agouti rats (male, 7-8 weeks old) were obtained from Harlan Laboratories (Maison-Alfort, France). Rats were kept on a 12 h light/dark cycle (0700-1900). Temperature was maintained at 22° C., and food and water were provided ad libitum.

### 3.1.3 Collagen Induced Arthritis (CIA)

One day before the experiment, CII solution (2 mg/mL) was prepared with 0.05 M acetic acid and stored at 4° C. Just before the immunization, equal volumes of adjuvant (IFA) and CII were mixed by a homogenizer in a pre-cooled glass bottle in an ice water bath. Extra adjuvant and prolonged homogenization may be required if an emulsion is not formed. 0.2 mL of the emulsion was injected intradermally at the base of the tail of each rat on day 1, a second booster intradermal injection (CII solution at 2 mg/mL in CFA 0.1 mL saline) was performed on day 9. This immunization method was modified from published methods (Sims et al, 2004; Jou et al., 2005).

### 3.1.4 Study Design

The therapeutic effects of the compounds were tested in the rat CIA model. Rats were randomly divided into equal groups and each group contained 10 rats. All rats were immunized on day 1 and boosted on day 9. Therapeutic dosing lasted from day 16 to day 30. The negative control group was treated with vehicle (MC 0.5%) and the positive control group with Enbrel (10 mg/kg, 3× week. s.c.). A compound of interest was typically tested at 5 doses, e.g. 1, 2, 3, 5, and 10 mg/kg, p.o.

### 3.1.5 Clinical Assessment of Arthritis

Arthritis is scored according to the method of Khachigian 2006, Lin et al 2007 and Nishida et al. 2004). The swelling of each of the four paws is ranked with the arthritic score as follows: O-no symptoms; 1-mild, but definite redness and swelling of one type of joint such as the ankle or wrist, or apparent redness and swelling limited to individual digits, regardless of the number of affected digits; 2-moderate redness and swelling of two or more types of joints; 3-severe redness and swelling of the entire paw including digits; 4-maximally inflamed limb with involvement of multiple joints (maximum cumulative clinical arthritis score 16 per animal) (Nishida et al., 2004).

To permit the meta-analysis of multiple studies the clinical score values were normalised as follows:

AUC of clinical score (AUC score): The area under the curve (AUC) from day 1 to day 14 was calculated for each individual rat. The AUC of each animal was divided by the average AUC obtained for the vehicle in the study from which the data on that animal was obtained and multiplied by 100 (i.e. the AUC was expressed as a percentage of the average vehicle AUC per study).

Clinical score increase from day 1 to day 14 (End point score): The clinical score difference for each animal was divided by the average clinical score difference obtained for the vehicle in the study from which the data on that animal was obtained and multiplied by 100 (i.e. the difference was expressed as a percentage of the average clinical score difference for the vehicle per study).

3.1.6 Change in Body Weight (%) after Onset of Arthritis

Clinically, body weight loss is associated with arthritis (Shelton et al., 2005; Rall, 2004; Walsmith et al., 2004). Hence, changes in body weight after onset of arthritis can be used as a non-specific endpoint to evaluate the effect of therapeutics in the rat model. The change in body weight (%) after onset of arthritis was calculated as follows:

$$Mice \text{:} \frac{Body {Weight}_{( week 6 )} - Body {Weight}_{( week 5 )}}{Body {Weight}_{( week 5 )}} \times 100 \%$$

$$Rats \text{:} \frac{Body {Weight}_{( week 4 )} - Body {Weight}_{( week 3 )}}{Body {Weight}_{( week 3 )}} \times 100 \%$$

### 3.1.7 Radiology

X-ray photos were taken of the hind paws of each individual animal. A random blind identity number was assigned to each of the photos, and the severity of bone erosion was ranked by two independent scorers with the radiological Larsen's score system as follows: 0—normal with intact bony outlines and normal joint space; 1—slight abnormality with any one or two of the exterior metatarsal bones showing slight bone erosion; 2—definite early abnormality with any three to five of the exterior metatarsal bones showing bone erosion; 3—medium destructive abnormality with all the exterior metatarsal bones as well as any one or two of the interior metatarsal bones showing definite bone erosions; 4—severe destructive abnormality with all the metatarsal bones showing definite bone erosion and at least one of the inner metatarsal joints completely eroded leaving some bony joint outlines partly preserved; 5—mutilating abnormality without bony outlines. This scoring system is a modification from Salvemini et al., 2001; Bush et al., 2002; Sims et al., 2004; Jou et al., 2005.

### 3.1.8 Histology

After radiological analysis, the hind paws of mice were fixed in 10% phosphate-buffered formalin (pH 7.4), decalcified with rapid bone decalcifiant for fine histology (Laboratories Eurobio) and embedded in paraffin. To ensure extensive evaluation of the arthritic joints, at least four serial sections (5 μm thick) were cut and each series of sections were 100 μm in between. The sections were stained with hematoxylin and eosin (H&E). Histologic examinations for synovial inflammation and bone and cartilage damage were performed double blind. In each paw, four parameters were assessed using a four-point scale. The parameters were cell infiltration, pannus severity, cartilage erosion and bone erosion. Scoring was performed according as follows: 1-normal, 2-mild, 3-moderate, 4-marked. These four scores are summed together and represented as an additional score, namely the ‘RA total score’.

3.1.9 Micro-Computed Tomography (uCT) Analysis of Calcaneus (Heel Bone):

Bone degradation observed in RA occurs especially at the cortical bone and can be revealed by μCT analysis (Sims N A et al., Arthritis Rheum. 50 (2004) 2338-2346: Targeting osteoclasts with zoledronic acid prevents bone destruction in collagen-induced arthritis; Oste L et al., ECTC Montreal 2007: A high throughput method of measuring bone architectural disturbance in a murine CIA model by micro-CT morphometry). After scanning and 3D volume reconstruction of the calcaneus bone, bone degradation is measured as the number of discrete objects present per slide, isolated in silico perpendicular to the longitudinal axis of the bone. The more the bone is degraded, the more discrete objects are measured. 1000 slices, evenly distributed along the calcaneus (spaced by about 10.8 μm), are analyzed.

### 3.1.10 Steady State PK

At day 7 or 11, blood samples were collected at the retro-orbital sinus with lithium heparin as anti-coagulant at the following time points: predose, 1, 3 and 6 h. Whole blood samples were centrifuged and the resulting plasma samples were stored at −20° C. pending analysis. Plasma concentrations of each test compound were determined by an LC-MS/MS method in which the mass spectrometer was operated in positive electrospray mode. Pharmacokinetic parameters were calculated using Winnonlin® (Pharsight®, United States) and it was assumed that the predose plasma levels were equal to the 24 h plasma levels.

### 3.1.10 Results

The compound of the invention displayed statistically significant efficacy from 3 mg/kg.

### 3.2 Septic Shock Model

Injection of lipopolysaccharide (LPS) induces a rapid release of soluble tumour necrosis factor (TNF-alpha) into the periphery. This model is used to analyse prospective blockers of TNF release in vivo.

Six BALB/cJ female mice (20 g) per group are treated at the intended dosing once, po. Thirty min later, LPS (15 mg/kg; E. Coli serotype 0111:B4) is injected ip. Ninety min later, mice are euthanized and blood is collected. Circulating TNF alpha levels are determined using commercially available ELISA kits. Dexamethasone (5 μg/kg) is used as a reference anti-inflammatory compound.

### 3.3 MAB Model

The MAB model allows a rapid assessment of the modulation of an RA-like inflammatory response by therapeutics (Kachigian L M. Nature Protocols (2006) 2512-2516: Collagen antibody-induced arthritis). DBA/J mice are injected i.v. with a cocktail of mAbs directed against collagen II. One day later, compound treatment is initiated (vehicle: 10% (v/v) HPIβCD). Three days later, mice receive an i.p. LPS injection (50 μg/mouse), resulting in a fast onset of inflammation. Compound treatment is continued until 10 days after the mAb injection. Inflammation is read by measuring paw swelling and recording the clinical score of each paw. The cumulative clinical arthritis score of four limbs is presented to show the severity of inflammation. A scoring system is applied to each limb using a scale of 0-4, with 4 being the most severe inflammation.
0 Symptom free

1 Mild, but definite redness and swelling of one type of joint such as the ankle or wrist, or apparent redness and swelling limited to individual digits, regardless of the number of affected digits
2 Moderate redness and swelling of two or more types of joints
3 Severe redness and swelling of the entire paw including digits
4 Maximally inflamed limb with involvement of multiple joints

### 3.4 Oncology Models

In vivo models to validate efficacy of small molecules towards JAK2-driven myeloproliferative diseases are described by Wernig et al. Cancer Cell 13, 311, 2008 and Geron et al. Cancer Cell 13, 321, 2008.

### 3.5 Mouse IBD Model

In vitro and in vivo models to validate efficacy of small molecules towards IBD are described by Wirtz et al. 2007.

### 3.6 Mouse Asthma Model

In vitro and in vivo models to validate efficacy of small molecules towards asthma are described by Nials et al., 2008; Ip et al. 2006; Pernis et al., 2002; Kudlacz et al., 2008.

### 3.7 Murine Model of Psoriatic-Like Epidermal Hyperplasia Induced by Intradermal Injections of IL22 or IL23

### 3.7.1 Materials

Mouse recombinant IL22 (582-ML-CF), carrier free is provided by R&D systems. Mouse recombinant IL23, carrier free (14-8231, CF) is provided by e-Bioscience.

### 3.7.2 Animals

Balb/c mice (female, 18-20 g body weight) are obtained from CERJ (France). Mice are kept on a 12 h light/dark cycle (07:00-19:00). Temperature is maintained at 22° C., food and water are provided ad libitum.

### 3.7.3 Study Design

The design of the study is adapted from Rizzo et al, 2011.

On the first day (D1), the mice are shaved around the two ears.

For 4 days (D1 to D4), the mice received a daily intradermal dose of mouse recombinant IL22 or IL23 (1 μg/20 μL in PBS/0.1% BSA) in the right pinna ear and 20 μL of PBS/0.1% BSA in the left pinna ear under anesthesia induced by inhalation of isoflurane.

From D1 to D5, mice are dosed with test-compound, G454627 (30 mg/kg, po, qd in MC0.5%), 1 hr prior IL23/IL22 injection or with vehicle.

### 3.7.4 Assessment of Disease

The thickness of both ears is measured daily with an automatic caliper. Body weight is assessed at initiation and at sacrifice. On fifth day, 2 hrs after the last dosing, the mice are sacrificed. The pinnae of the ear are cut, excluding cartilage. The pinnae are weighed and then, placed in vial containing 1 mL of RNAlater solution or in formaldehyde.

There are 8 mice per group. The results are expressed as mean±sem and statistical analysis is performed using one-way Anova followed by Dunnett's post-hoc test versus IL22 or IL23 vehicle groups.

### 3.7.5 Histology

After sacrifice, ears are collected and fixed in 3.7% formaldehyde before embedding in paraffin. Two μm thick sections are done and stained with hematoxylin and eosin. Ear epidermis thickness is measured by image analysis (Sis'Ncom software) with 6 images per ear captured at magnification ×20. Data are expressed as mean±sem and statistical analysis is performed using one-way Anova followed by Dunnett's post-hoc test versus IL22 or IL23 vehicle groups.

### 3.7.6 RNA Extraction, RT-PCR and Real-Time PCR

IL-17a, IL-22, IL-1β, LCN2 and S100A9 transcript levels in ear tissue are determined using real-time quantitative PCR.

### Example 4

### Pharmacokinetic, ADME and Toxicity Assays

### 4.1 Thermodynamic Solubility

Test compound is added to 0.2M phosphate buffer pH 7.4 or 0.1M citrate buffer pH 3.0 at a concentration of 1 mg/mL in a glass vial.

The samples are rotated in a Rotator drive STR 4 (Stuart Scientific, Bibby) at speed 3.0 at room temperature for 24 h.

After 24 h, 800 μL of the sample is transferred to an eppendorf tube and centrifuged 5 min at 14000 rpm. 200 μL of the supernatant of the sample is then transferred to a MultiscreenR Solubility Plate (Millipore, MSSLBPC50) and the supernatant is filtered (10-12″ Hg) with the aid of a vacuum manifold into a clean Greiner polypropylene V-bottom 96 well plate (Cat no. 651201). 5 μL of the filtrate is diluted into 95 μL (F20) of the same buffer used to incubate in the plate containing the standard curve (Greiner, Cat no. 651201).

The standard curve for the compound is prepared freshly in DMSO starting from a 10 mM DMSO stock solution diluted factor 2 in DMSO (5000 μM) and then further diluted in DMSO up to 19.5 μM. 3 μL of the dilution series as from 5000 μM is then transferred to a 97 μL acetonitrile-buffer mixture (50/50). The final concentration range is 2.5 to 150 μM.

The plate is sealed with sealing mats (MA96RD-045, www.kinesis.co.uk) and samples are measured at room temperature on LCMS (ZQ 1525 from Waters) under optimized conditions using Quanoptimize to determine the appropriate mass of the molecule.

The samples are analyzed on LCMS with a flow rate of 1 mL/min. Solvent A is 15 mM ammonia and solvent B is acetonitrile. The sample is run under positive ion spray on an XBridge C18 3.5 μM (2.1×30 mm) column, from Waters. The solvent gradient has a total run time of 2 min and ranges from 5% B to 95% B.

Peak areas are analyzed with the aid of Masslynx software package and peak areas of the samples are plotted against the standard curve to obtain the solubility of the compound.

Solubility values are reported in μM or μg/mL.

### 4.2 Aqueous Solubility

Starting from a 10 mM stock in DMSO, a serial dilution of the compound is prepared in DMSO. The dilution series is transferred to a 96 NUNC Maxisorb plate F-bottom (Cat no. 442404) and 0.1M phosphate buffer pH7.4 or 0.1M citrate buffer pH3.0 at room temperature is added.

The final concentration ranges from 300 μM to 18.75 μM in 5 equal dilution steps. The final DMSO concentration does not exceed 3%. 200 μM Pyrene is added to the corner points of each 96 well plate and serves as a reference point for calibration of Z-axis on the microscope.

The assay plates are sealed and incubated for 1 h at 37° C. while shaking at 230 rpm. The plates are then scanned under a white light microscope, yielding individual pictures of the precipitate per concentration. The precipitate is analyzed and converted into a number with a software tool which can be plotted onto a graph. The first concentration at which the compound appears completely dissolved is the concentration reported; however the true concentration lies somewhere between this concentration and one dilution step higher.

Solubility values measured according to this protocol are reported in μg/mL.

### 4.3 Plasma Protein Binding (Equilibrium Dialysis)

A 10 mM stock solution of the compound in DMSO is diluted with a factor 5 in DMSO. This solution is further diluted in freshly thawed human, rat, mouse or dog plasma (BioReclamation INC) with a final concentration of 5 μM and final DMSO concentration of 0.5% (5.5 μL in 1094.5 μL plasma in a PP-Masterblock 96 well (Greiner, Cat no. 780285))

A Pierce Red Device plate with inserts (ThermoScientific, Cat no. 89809) is prepared and filled with 750 μL PBS in the buffer chamber and 500 μL of the spiked plasma in the plasma chamber. The plate is incubated for 4 h at 37° C. while shaking at 230 rpm. After incubation, 120 μL of both chambers is transferred to 360 μL acetonitrile in a 96-well round bottom, PP deep-well plates (Nunc, Cat no. 278743) and sealed with an aluminum foil lid. The samples are mixed and placed on ice for 30 min. This plate is then centrifuged 30 min at 1200 rcf at 4° C. and the supernatant is transferred to a 96 v-bottom PP plate (Greiner, 651201) for analysis on LCMS.

The plate is sealed with sealing mats (MA96RD-04S) of www.kinesis.co.uk and samples are measured at room temperature on LCMS (ZQ 1525 from Waters) under optimized conditions using Quanoptimize to determine the appropriate mass of the molecule.

The samples are analyzed on LCMS with a flow rate of 1 mL/min. Solvent A is 15 mM ammonia and solvent B is acetonitrile. The sample is run under positive ion spray on an XBridge C18 3.5 μM (2.1×30 mm) column, from Waters. The solvent gradient has a total run time of 2 min and ranges from 5% B to 95% B.

Peak area from the compound in the buffer chamber and the plasma chamber are considered to be 100% compound. The percentage bound to plasma is derived from these results and is reported as percentage bound to plasma.

The solubility of the compound in the final test concentration in PBS is inspected by microscope to indicate whether precipitation is observed or not.

### 4.4 Caco2 Permeability

Bi-directional Caco-2 assays are performed as described below. Caco-2 cells are obtained from European Collection of Cell Cultures (ECACC, cat 86010202) and used after a 21 day cell culture in 24-well Transwell plates (Fisher TKT-545-020B).

2×10⁵ cells/well are seeded in plating medium consisting of DMEM+GlutaMAXI+1% NEAA+10% FBS (FetalClone II)+1% Penicillin/Streptomycin. The medium is changed every 2-3 days.

Test and reference compounds (propranolol and rhodamine-123 or vinblastine, all purchased from Sigma) are prepared in Hanks' Balanced Salt Solution containing 25 mM HEPES (pH7.4) and added to either the apical (125 μL) or basolateral (600 μL) chambers of the Transwell plate assembly at a concentration of 10 μM with a final DMSO concentration of 0.25%.

50 μM Lucifer Yellow (Sigma) is added to the donor buffer in all wells to assess integrity of the cell layers by monitoring Lucifer Yellow permeation. As Lucifer Yellow (LY) cannot freely permeate lipophilic barriers, a high degree of LY transport indicates poor integrity of the cell layer.

After a 1 h incubation at 37° C. while shaking at an orbital shaker at 150 rpm, 70 μL aliquots are taken from both apical (A) and basal (B) chambers and added to 100 μL150:50 acetonitrile:water solution containing analytical internal standard (0.5 μM carbamazepine) in a 96 well plate.

Lucifer yellow is measured with a Spectramax Gemini XS (Ex 426 nm and Em 538 nm) in a clean 96 well plate containing 150 μL of liquid from basolateral and apical side.

Concentrations of compound in the samples are measured by high performance liquid-chromatography/mass spectroscopy (LC-MS/MS).

Apparent permeability (Pₐₚₑ) values are calculated from the relationship:

$${P}_{app} = {[ compound ]}_{acceptor final} \times {V}_{acceptor} / ( {[ compound ]}_{donor initial} \times {V}_{donor} ) \times 60 \times {10}^{- 6} cm \text{/} s$$

V=chamber volume=
T_{inc}=incubation time.
Surface area=0.33 cm²

The Efflux ratios, as an indication of active efflux from the apical cell surface, are calculated using the ratio of PₐₚₚB>A/PₐₚₚA>B.

The following assay acceptance criteria are used:
Propranolol: Pₐₚₚ(A>B) value≧20(×10⁻⁶ cm/s)
Rhodamine 123 or Vinblastine: Pₐₚₚ(A>B) value<5 (×10⁻⁶ cm/s) with Efflux ratio≧5.
Lucifer yellow permeability: ≦100 nm/s

### 4.5 MDCKII-MDR1 Permeability

MDCKII-MDR1 cells are Madin-Darby canine kidney epithelial cells, over-expressing human multi-drug resistance (MDR1) gene, coding for P-glycoprotein (P-gp). Cells are obtained from Netherlands Cancer Institute and used after a 3-4 day cell culture in 24-well Millicell cell culture insert plates (Millipore, PSRP010R5). Bi-directional MDCKII-MDR1 permeability assay is performed as described below.

3×10⁵ cells/mL (1.2×10⁵ cells/well) are seeded in plating medium consisting of DMEM+1% Glutamax-100+1% Antibiotic/Antimycotic+10% FBS (Biowest, 51810). Cells are left in CO₂ incubator for 3-4 days. The medium is changed 24 h after seeding and on the day of experiment.

Test and reference compounds (amprenavir and propranolol) are prepared in Dulbecco's phosphate buffer saline (D-PBS, pH7.4) and added to either the apical (400 μL) or basolateral (800 μL) chambers of the Millicell cell culture insert plates assembly at a final concentration of 10 μM (0.5 μM in case of amprenavir) with a final DMSO concentration of 1%.

100 μM Lucifer Yellow (Sigma) is added to the all donor buffer solutions, in order to assess integrity of the cell monolayers by monitoring Lucifer Yellow permeation. Lucifer yellow is a fluorescent marker for the paracellular pathway and it is used as an internal control in every monolayer to verify tight junction integrity during the assay.

After a 1 h incubation at 37° C. while shaking at an orbital shaker at 150 rpm, 75 μL aliquots are taken from both apical (A) and basal (B) chambers and added to 225 μL acetonitrile:water solution (2:1) containing analytical internal standard (10 ng/mL warfarin) in a 96 well plate. Aliquoting is also performed at the beginning of the experiment from donor solutions to obtain initial (Co) concentration.

Concentration of compound in the samples is measured by high performance liquid-chromatography/mass spectroscopy (LC-MS/MS).

Lucifer yellow is measured with a Fluoroscan Ascent FL Thermo Scientific (Ex 485 nm and Em 530 nm) in a 96 well plate containing 150 μL of liquid from all receiver wells (basolateral or apical side).

### 4.6 Liver Microsomal Stability

A 10 mM stock solution of compound in DMSO is diluted to 6 μM in a 105 mM phosphate buffer, pH7.4 in a 96 deep well plate (Greiner, Cat no. 780285) and pre-warmed at 37° C.

A Glucose-6-phosphate-dehydrogenase (G6PDH, Roche, 10127671001) working stock solution of 700 U/mL is diluted with a factor 1:700 in a 105 mM phosphate buffer, pH7.4. A co-factor mix containing 0.528M MgCl₂.6H₂O (Sigma, M2670), 0.528M glucose-6-phosphate (Sigma, G-7879) and 0.208M NADP+ (Sigma, N-0505) is diluted with a factor 1:8 in a 105 mM phosphate buffer, pH7.4.

A working solution is made containing 1 mg/mL liver microsomes (Provider, Xenotech) of the species of interest (human, mouse, rat, dog . . . ), 0.8 U/mL G6PDH and co-factor mix (6.6 mM MgCl₂, 6.6 mM glucose-6-phosphate, 2.6 mM NADP+). This mix is pre-incubated for 15 min, but never more than 20 min, at room temperature.

After pre-incubation, compound dilution and the mix containing the microsomes, are added together in equal amount and incubated for 30 min at 300 rpm. For the time point of 0 min, two volumes of MeOH are added to the compound dilution before the microsome mix is added. The final concentration during incubation are: 3 μM test compound or control compound, 0.5 mg/mL microsomes, 0.4 U/mL G6PDH, 3.3 mM MgCl₂, 3.3 mM glucose-6-phosphate and 1.3 mM NaDP+.

After 30 min of incubation, the reaction is stopped with 2 volumes of MeOH.

At both time points, samples are mixed, centrifuged and the supernatant is harvested for analysis on LC-MS/MS. The instrument responses (i.e. peak heights) are referenced to the zero time-point samples (as 100%) in order to determine the percentage of compound remaining Standard compounds Propanolol and Verapamil are included in the assay design.

The data on microsomal stability are expressed as a percentage of the total amount of compound remaining after 30 min.

### 4.7 Hepatocyte Stability

Models to evaluate metabolic clearance in hepatocyte are described by McGinnity et al. Drug Metabolism and Disposition 2008, 32, 11, 1247.

### 4.8 Pharmacokinetic Study in Rodents

### 4.8.1 Animals

Sprague-Dawley rats (male, 5-6 weeks old) are obtained from Janvier (France). Rats are acclimatized for at least 7 days before treatment and are kept on a 12 h light/dark cycle (0700-1900). Temperature is maintained at approximately 22° C., and food and water are provided ad libitum. Two days before administration of the test compounds, rats underwent surgery to place a catheter in the jugular vein under isoflurane anesthesia. After the surgery, rats are housed individually. Rats are deprived of food for at least 16 h before oral dosing and 6 h after. Water is provided ad libitum.

### 4.8.2 Pharmacokinetic Study

Compounds are formulated in PEG200/physiological saline (60/40) for the intravenous route and in 0.5% methylcellulose and 10% hydroxylpropyl-β-cyclodextrine pH 3 for the oral route. Test compounds are orally dosed as a single esophageal gavage at 5 mg/kg under a dosing volume of 5 mL/kg and intravenously dosed as a bolus via the caudal vein at 1 mg/kg under a dosing volume of 5 mL/kg. Each group consisted of 3 rats. Blood samples are collected via the jugular vein with lithium heparin as anti-coagulant at the following time points: 0.05, 0.25, 0.5, 1, 3, 5 and 8 h (intravenous route), and 0.25, 0.5, 1, 3, 5, 8 and 24 h (oral route). Alternatively, blood samples are collected at the retro-orbital sinus with lithium heparin as anti-coagulant at the following time points 0.25, 1, 3 and 6 h (oral route). Whole blood samples are centrifuged at 5000 rpm for 10 min and the resulting plasma samples are stored at −20° C. pending analysis.

### 4.8.3 Quantification of Compound Levels in Plasma

Plasma concentrations of each test compound are determined by an LC-MS/MS method in which the mass spectrometer is operated in positive electrospray mode.

### 4.8.4 Determination of Pharmacokinetic Parameters

Pharmacokinetic parameters are calculated using Winnonlin® (Pharsight®, United States).

### 4.9 7-Day Rat Toxicity Study

A 7-day oral toxicity study with test compounds is performed in Sprague-Dawley male rats to assess their toxic potential and toxicokinetics, at daily doses of 100, 300 and 500 mg/kg/day, by gavage, at the constant dosage-volume of 5 mL/kg/day.

The test compounds are formulated in 30% (v/v) HPβCD in purified water. Each group included 5 principal male rats as well as 3 satellite animals for toxicokinetics. A fourth group is given 30% (v/v) HPβCD in water only, at the same frequency, dosage volume and by the same route of administration, and acted as the vehicle control group.

The goal of the study is to determine the lowest dose that resulted in no adverse events being identified (no observable adverse effect level—NOAEL).

### 4.10 Liability for QT Prolongation

Potential for QT prolongation is assessed in the hERG patch clamp assay.

### 4.11 Conventional Whole-Cell Patch-Clamp

Whole-cell patch-clamp recordings are performed using an EPC10 amplifier controlled by Pulse v8.77 software (HEKA). Series resistance is typically less than 10 MΩ and compensated by greater than 60%, recordings are not leak subtracted. Electrodes are manufactured from GC150TF pipette glass (Harvard).

The external bathing solution contained: 135 mM NaCl, 5 mM KCl, 1.8 mM CaCl₂, 5 mM Glucose, 10 mM HEPES, pH 7.4.

The internal patch pipette solution contained: 100 mM Kgluconate, 20 mM KCl, 1 mM CaCl₂, 1 mM MgCl₂, 5 mM Na₂ATP, 2 mM Glutathione, 11 mM EGTA, 10 mM HEPES, pH 7.2.

Drugs are perfused using a Biologic MEV-9/EVH-9 rapid perfusion system.

All recordings are performed on HEK293 cells stably expressing hERG channels. Cells are cultured on 12 mm round coverslips (German glass, Bellco) anchored in the recording chamber using two platinum rods (Goodfellow). hERG currents are evoked using an activating pulse to +40 mV for 1000 ms followed by a tail current pulse to −50 mV for 2000 ms, holding potential is −80 mV. Pulses are applied every 20 s and all experiments are performed at room temperature.

### GENERAL CONCLUSIONS

The data provided in the present application demonstrate that Compound I (the compound of the invention) exhibits in vitro and in vivo potency. Moreover, Compound I exhibits a high selectivity of at least 10 fold vs the other JAK family members (JAK2, JAK3, and TYK2). In particular, Compound I inhibits JAK1 with a >15 fold selectivity vs JAK2, >70 fold selectivity vs JAK3, and >80 fold vs TYK2. Such selectivity is expected to result in a good safety profile, in particular with respect to side-effects that may occur via off-target activity.

### REFERENCES

Bundgard, H., 1985 Design of Prodrugs, pp. 7-9, 21-24, Elsevier, Amsterdam.
Bush K. A. et al. 2002 Arthritis Rheum. 46: 802-5.
Choy E. H. et al. 2001 N Engl J. Med. 344: 907-16.
Chubinskaya S. et al. 2003 The international journal of biochemistry & cell biology 35(9)1323-1340.
Clegg D. O. et al. 2006 N Engl J. Med. 2006 354:795-808.
Constantinescu et al. 2007 Trends in Biochemical Sciences 33(3): 122-131.
Firestein G. S. 2003 Nature. 423:356-61.
Geron et al. 2008 Cancer Cell 13 (4), 321-30
Ip et al. 2006 Clin. Exp. Immun, 162-172.
Jou I M, et al. 2005 Arthritis Rheum. 52:339-44.
Khachigian, L. M. et al. 2006 Nature Protocols 1, 2512-6.
Kopf et al. 2010 Nat. Rev. Drug Disc., 703-718.
Kudlacz et al. 2008 Eur J Pharmaco 154-161.
Lee D. M. et al. 2001 Lancet. 358: 903-11.
Legendre F et al. 2003 J Biol. Chem. 278(5)2903-2912.
Levy D. et al. New England Journal of Medicine 2007 357 1655-1658
Li W. Q. et al. 2001 J Immunol 166:3491-3498.
Lin H. S. et al. 2007 Br J. Pharmacol. April; 150 (7):829-31.
McGinnity et al. 2008 Drug Metabolism and Disposition, 32, 11, 1247.
Mullighan C. G. et al. 2009, PNAS 1, 23,9414-9418.
Nials et al. 2008 Disease Models & Mechanisms, 213-220.
Nishida K et al. 2004 Arthritis Rheum. 10: 3365-76.
O'Shea J. et al. 2004 Nature Review Drug Discovery 3, 555-564.
O'Sullivan et al. 2007 Mol Immunol 44(10):2497-506.
O'Dell J R. et al. 2004 N Engl J. Med. 350(25):2591-602.
Osaki M et al. 2003 Biochem J 369:103-115.
Otero M et al. 2005 Arthritis Research & Therapy 7:R581-R591.
Pernis et al. 2002 J. Clin. Invest. 1279.
Rall L C et al. 2004 Rheumatology; 10:1219-23.
Remington's Pharmaceutical Sciences, 17th edition, 1985, Mack Publishing Company, Easton, Pa. 50 2338-2346.
Rodig S. J. et al. 1998 Cell 93: 373-383.
Salvemini D et al. 2001 Arthritis Rheum. 44:2909-21.
Shelton D. L. et al. 2005 Pain. 116:8-16.
Sims N A et al. 2004 Arthritis Rheum. 50 2338-2346.
Smolen J. S. et al. 2003. Nat Rev Drug Discov. 2: 473-88.
T. W. Greene and P. G. M. Wuts, 1991 Protecting Groups in Organic Synthesis, Second Edition, Wiley, New York.
Tam, L. et al. 2007 British Journal of Cancer, 97, 378-383.
Tetsuji Naka et al. 2002 Arthritis Res, 4 (suppl 3):5233-5242.
Vainchenker W. et al. 2008 Seminars in Cell & Developmental Biology 19, 385-393.
Vandeghinste et al. WO 2005/124342.
Walsmith J et al. 2004 J. Rheumatol.; 31:23-9.
Wernig et al. 2008 Cancer Cell 13(4), 311-320.
Wieland H A et al. 2005 Nat Rev Drug Discov. 4:331-44.
Wirtz et al. 2007 Advanced Drug Delivery Reviews, 2007, 1073-1083.
Xiang, 2008, Blood, 111, 9, 4809.
Kan Z. et al. Genome Res 2013; 23:1422-33.
Staerk J. et al. J Biol Chem 2005; 280:41893-41899.
Zenatti P. P. et al. Nat. Gen. 2011; 43:932-41.
Collins M. K. L. et al, J Exp Med. 1992, 176, 1043-1051.

### FINAL REMARKS

All publications, including but not limited to patents and patent applications, cited in this specification are herein incorporated by reference as if each individual publication were specifically and individually indicated to be incorporated by reference herein as though fully set forth.

From the foregoing description, various modifications and changes in the compositions and methods of this invention will occur to those skilled in the art. All such modifications coming within the scope of the appended claims are intended to be included therein. It will be appreciated by those skilled in the art that the foregoing descriptions are exemplary and explanatory in nature, and intended to illustrate the invention and its preferred embodiments. Through routine experimentation, an artisan will recognise apparent modifications and variations that may be made without departing from the spirit of the invention. Thus, the invention is intended to be defined not by the above description, but by the following claims and their equivalents.

It should be understood that factors such as the differential cell penetration capacity of the various compounds can contribute to discrepancies between the activity of the compounds in the in vitro biochemical and cellular assays.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using either ChemDraw® or ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

1. A compound according to Formula I: 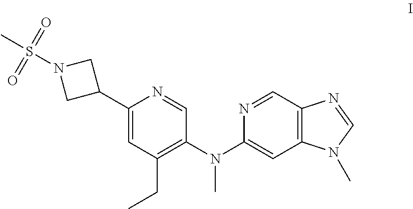 [SMILES: CCc1cc(C2CN(S(C)(=O)=O)C2)ncc1N(C)c1cc2c(cn1)ncn2C]
or a pharmaceutically acceptable salt, or a solvate, or a solvate of the pharmaceutically acceptable salts.

2. The compound according to claim 1, wherein the compound is according to Formula I.

3. A pharmaceutical composition comprising a pharmaceutically acceptable carrier and a pharmaceutically effective amount of the compound according to claim 1.

4. The pharmaceutical composition according to claim 3 comprising a further therapeutic agent.

5. (canceled)

6. (canceled)

7. (canceled)

8. (canceled)

9. A method for the treatment or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, comprising administering an amount of the compound according to claim 1, sufficient to effect said treatment or prophylaxis.

10. The method according to claim 9, wherein said compound is administered in combination with a further therapeutic agent.

11. The pharmaceutical composition according to claim 4, wherein the further therapeutic agent is an agent for the treatment or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

12. The method according to claim 10, wherein the further therapeutic agent is an agent for the treatment or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons.

13. A method for the treatment or prophylaxis of allergic or inflammatory conditions, autoimmune diseases, proliferative diseases, transplantation rejection, diseases involving impairment of cartilage turnover, congenital cartilage malformations, and/or diseases associated with hypersecretion of IL6 or interferons, comprising administering an amount of a pharmaceutical composition according to claim 3, sufficient to effect said treatment or prophylaxis.

14. The method according to claim 13, wherein the pharmaceutical composition according to claim 3 is administered in combination with a further therapeutic agent.

15. The method according to claim 14, wherein the further therapeutic agent is an agent for the treatment, prevention or prophylaxis of inflammatory conditions, autoimmune diseases, proliferative diseases, transplantation rejection, diseases involving impairment of cartilage turnover, congenital cartilage malformations, and/or diseases associated with hypersecretion of IL6.
